(19) United States
(12) Patent Application Publication
(10) Pub. No.: US 2022/0019146 A1
(43) Pub. Date: Jan. 20, 2022

(54) FILM FORMING MATERIAL FOR LITHOGRAPHY, COMPOSITION FOR FILM FORMATION FOR LITHOGRAPHY, UNDERLAYER FILM FOR LITHOGRAPHY, AND METHOD FOR FORMING PATTERN

(71) Applicant: Mitsubishi Gas Chemical Company, Inc., Tokyo (JP)

(72) Inventors: Masahiro YAMANE, Kurashiki-shi, Okayama (JP); Kouichi YAMADA, Kurashiki-shi, Okayama (JP); Junya HORIUCHI, Hiratsuka-shi, Kanagawa (JP); Takashi MAKINOSHIMA, Hiratsuka-shi, Kanagawa (JP); Masatoshi ECHIGO, Chiyoda-ku, Tokyo (JP)

(21) Appl. No.: 17/295,790

(22) PCT Filed: Nov. 21, 2019
(86) PCT No.: PCT/JP2019/045544

§ 371 Date: May 20, 2021

(30) Foreign Application Priority Data
Nov. 21, 2018 (JP) 2018-218042

(51) Int. Cl. G03F 7/11; C07D 207/452; C08G 73/12; C09D 179/08

(52) U.S. Cl. CPC G03F 7/11; C09D 179/085; C08G 73/12; C07D 207/452

(57) ABSTRACT

The present invention provides a film forming material for lithography comprising a compound having:
a group of formula (0A):

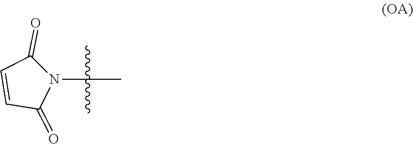 [SMILES: *N1C(=O)C=CC1=O]

and
a group of formula (0B):

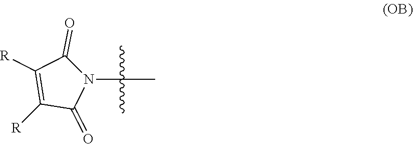

wherein
each R is independently selected from the group consisting of a hydrogen atom and an alkyl group having 1 to 4 carbon atoms, provided that at least one R is an alkyl group having 1 to 4 carbon atoms.

### TECHNICAL FIELD

The present invention relates to a film forming material for lithography, a composition for film formation for lithography containing the material, an underlayer film for lithography formed by using the composition, and a method for forming a pattern (for example, a method for forming a resist pattern or a circuit pattern) by using the composition.

### BACKGROUND ART

In the production of semiconductor devices, fine processing is practiced by lithography using photoresist materials. In recent years, further miniaturization based on pattern rules has been demanded along with increase in the integration and speed of LSI. And now, lithography using light exposure, which is currently used as a general purpose technique, is approaching the limit of essential resolution derived from the wavelength of a light source.

The light source for lithography used upon forming resist patterns has been shifted to ArF excimer laser (193 nm) having a shorter wavelength from KrF excimer laser (248 nm). However, when the miniaturization of resist patterns proceeds, the problem of resolution or the problem of collapse of resist patterns after development arises. Therefore, resists have been desired to have a thinner film. Nevertheless, if resists merely have a thinner film, it is difficult to obtain the film thicknesses of resist patterns sufficient for substrate processing. Therefore, there has been a need for a process of preparing a resist underlayer film between a resist and a semiconductor substrate to be processed, and imparting functions as a mask for substrate processing to this resist underlayer film in addition to a resist pattern.

Various resist underlayer films for such a process are currently known. For example, as a material for realizing resist underlayer films for lithography having the selectivity of a dry etching rate close to that of resists, unlike conventional resist underlayer films having a fast etching rate, an underlayer film forming material for a multilayer resist process containing a resin component having at least a substituent that generates a sulfonic acid residue by eliminating a terminal group under application of predetermined energy, and a solvent has been suggested (see Patent Literature 1). Moreover, as a material for realizing resist underlayer films for lithography having the selectivity of a dry etching rate smaller than that of resists, a resist underlayer film material comprising a polymer having a specific repeat unit has been suggested (see Patent Literature 2). Furthermore, as a material for realizing resist underlayer films for lithography having the selectivity of a dry etching rate smaller than that of semiconductor substrates, a resist underlayer film material comprising a polymer prepared by copolymerizing a repeat unit of an acenaphthylene and a repeat unit having a substituted or unsubstituted hydroxy group has been suggested (see Patent Literature 3).

Meanwhile, as materials having high etching resistance for this kind of resist underlayer film, amorphous carbon underlayer films formed by CVD using methane gas, ethane gas, acetylene gas, or the like as a raw material are well known.

In addition, the present inventors have suggested an underlayer film forming composition for lithography containing a naphthalene formaldehyde polymer comprising a particular structural unit and an organic solvent (see Patent Literatures 4 and 5) as a material that is not only excellent in optical properties and etching resistance, but also is soluble in a solvent and applicable to a wet process.

As for methods for forming an intermediate layer used in the formation of a resist underlayer film in a three-layer process, for example, a method for forming a silicon nitride film (see Patent Literature 6) and a CVD formation method for a silicon nitride film (see Patent Literature 7) are known. Also, as intermediate layer materials for a three-layer process, materials comprising a silsesquioxane-based silicon compound are known (see Patent Literatures 8 and 9).

Patent Literature 10 discloses a photosensitive resin composition comprising (A) an alkali soluble binder polymer, (B) a photopolymerizable compound, (C) a photopolymerization initiator, and (D) a maleic acid derivative, and a polymaleimide represented by formula (5a) is mentioned as one example of the (D) maleic acid derivative. The above photosensitive resin composition is said to be excellent in all characteristics of sensitivity, resolution, and adhesiveness with the substrate.

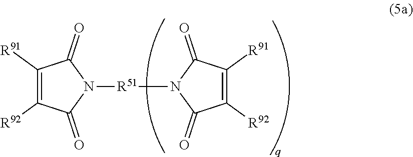

[In the formula, R⁵¹ represents a single bond, or a divalent organic group composed of at least one group selected from the group consisting of an alkylene group, an arylene group, an oxy group, a carbonyl group, an ester group, a carbonate group, and a urethane group; R⁹¹ and R⁹² each independently represent a hydrogen atom, an alkyl group having 1 to 10 carbon atoms, a cycloalkyl group having 3 to 10 carbon atoms, an aryl group, an alkoxy group, or a halogen atom; q is an integer of 1 to (the number of bonding hands of R⁵¹ that can be bonded); and alternatively, R⁹¹ and R⁹² are optionally taken together with the carbon atoms at position 3 and position 4 of the imide group to form a divalent group constituting a 5-membered ring or 6-membered ring structure.]

Patent Literature 11 discloses a resin composition comprising (A) a cyanic acid ester compound and (B) a bismaleimide compound represented by the following formula (1), and it is said that such a resin composition can realize a printed wiring board that is excellent in heat resistance, peel strength, and thermal expansion coefficient.

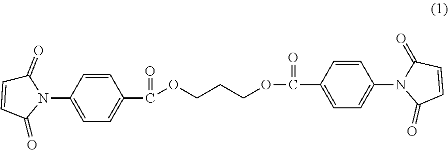 [SMILES: O=C(OCCCOC(=O)c1ccc(N2C(=O)C=CC2=O)cc1)c1ccc(N2C(=O)C=CC2=O)cc1]

Patent Literature 12 discloses a thermosetting resin composition comprising (A) a carboxyl group-containing modified ester resin, (B) a compound that is at least one selected from the group consisting of an epoxy group-containing compound, an isocyanate group-containing compound, and a blocked isocyanate group-containing compound, and (C) a thermosetting aid. Also, a maleimide compound and a citraconimide compound are mentioned as an example of the above thermosetting aid (C). The above photosensitive resin composition is said to be very excellent in terms of adhesive properties, heat resistance, flexibility, bendability, adhesiveness, electrical insulation properties, moist heat resistance, and the like, especially in terms of compatibility of adhesive properties and electrical insulation properties, and compatibility of bendability and heat resistance.

### CITATION LIST

### Patent Literature

Patent Literature 1: Japanese Patent Application Laid-Open No. 2004-177668
Patent Literature 2: Japanese Patent Application Laid-Open No. 2004-271838
Patent Literature 3: Japanese Patent Application Laid-Open No. 2005-250434
Patent Literature 4: International Publication No. WO 2009/072465
Patent Literature 5: International Publication No. WO 2011/034062
Patent Literature 6: Japanese Patent Application Laid-Open No. 2002-334869
Patent Literature 7: International Publication No. WO 2004/066377
Patent Literature 8: Japanese Patent Application Laid-Open No. 2007-226170
Patent Literature 9: Japanese Patent Application Laid-Open No. 2007-226204
Patent Literature 10: Japanese Patent Application Laid-Open No. 2005-141084
Patent Literature 11: Japanese Patent Application Laid-Open No. 2017-071738
Patent Literature 12: Japanese Patent Application Laid-Open No. 2012-131967

### SUMMARY OF INVENTION

### Technical Problem

As mentioned above, a large number of film forming materials for lithography have heretofore been suggested. However, none of these materials not only have high solvent solubility that permits application of a wet process such as spin coating or screen printing but also achieve all of heat resistance, etching resistance, embedding properties to a substrate having difference in level, and film flatness at high dimensions. Thus, the development of novel materials is required.

Patent Literature 10 discloses the use of the polymaleimide represented by formula (5a), Patent Literature 11 discloses the use of the bismaleimide compound represented by formula (1), and Patent Literature 12 discloses the use of a maleimide compound and a citraconimide compound. However, none of these literatures offer a guideline for obtaining a film forming material for lithography that is applicable to a wet process, and is useful for forming a photoresist underlayer film excellent in heat resistance, etching resistance, embedding properties to a substrate having difference in level, and film flatness.

The present invention has been made in light of the problems described above, and an object of the present invention is to provide a film forming material for lithography that is applicable to a wet process, and is useful for forming a photoresist underlayer film excellent in heat resistance, etching resistance, embedding properties to a substrate having difference in level, and film flatness; a composition for film formation for lithography comprising the material; as well as an underlayer film for lithography and a method for forming a pattern by using the composition.

### Solution to Problem

The present inventors have, as a result of devoted examinations to solve the above problems, found out that use of a compound having a specific structure can solve the above problems, and reached the present invention. More specifically, the present invention is as follows.
[1]

A film forming material for lithography comprising a compound having:

a group of formula (0A):

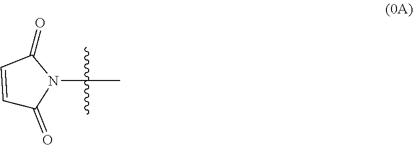 [SMILES: *N1C(=O)C=CC1=O]

and

a group of formula (0B):

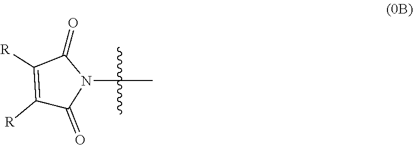

(In formula (0B), each R is independently selected from the group consisting of a hydrogen atom and an alkyl group having 1 to 4 carbon atoms, provided that at least one R is an alkyl group having 1 to 4 carbon atoms.)
[2]

The film forming material for lithography according to [1], wherein the compound is represented by formula (1A₀).

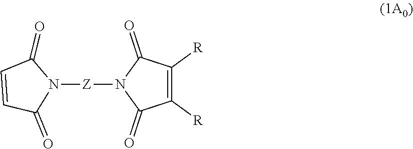

(In formula (1A₀),

each R is independently selected from the group consisting of a hydrogen atom and an alkyl group having 1 to 4 carbon atoms, provided that at least one R is an alkyl group having 1 to 4 carbon atoms; and

Z is a divalent group having 1 to 100 carbon atoms and optionally containing a heteroatom.)
[3]

The film forming material for lithography according to [1] or [2], wherein the compound is represented by formula (1A).

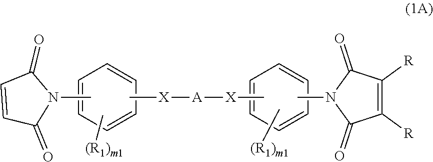

(In formula (1A),

each R is independently selected from the group consisting of a hydrogen atom and an alkyl group having 1 to 4 carbon atoms, provided that at least one R is an alkyl group having 1 to 4 carbon atoms;

each X is independently selected from the group consisting of a single bond, —O—, —CH₂—, —C(CH₃)₂—, —CO—, —C(CF₃)₂—, —CONH—, and —COO—;

A is selected from the group consisting of a single bond, an oxygen atom, and a divalent group having 1 to 80 carbon atoms and optionally containing a heteroatom;

each R₁ is independently a group having 0 to 30 carbon atoms and optionally containing a heteroatom; and each ml is independently an integer of 0 to 4.)
[4]

The film forming material for lithography according to [3], wherein:

A is a single bond, an oxygen atom, —(CH₂)ₚ—, —CH₂C (CH₃)₂CH₂—, —(C(CH₃)₂)ₚ—, —(O(CH₂)_{q})ₚ—, —(O(C₆H₄))ₚ—, or any of the following structures:

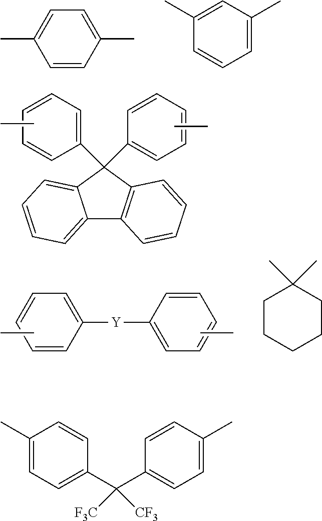

Y is a single bond, —O—, —CH2—, —C(CH₃)₂—, —C(CF₃)₂—,

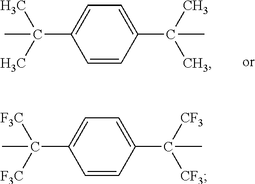

p is an integer of 0 to 20; and

q is an integer of 0 to 4.
[5]

The film forming material for lithography according to [3] or [4], wherein:

each X is independently a single bond, —O—, —C(CH₃)₂—, —CO—, or —COO—;

A is a single bond, an oxygen atom, or the following structures:

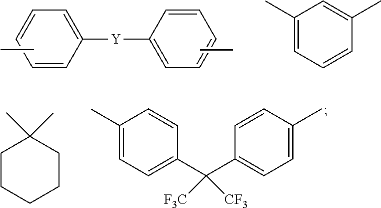

and

Y is —C(CH₃)₂— or —C(CF₃)₂—.
[6]

The film forming material for lithography according to [1] or [2], wherein the compound is represented by formula (2A).

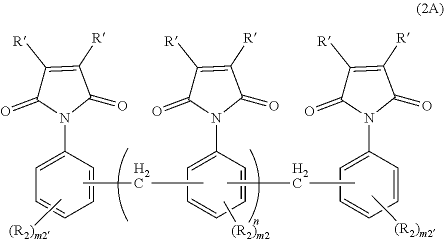

(In formula (2A),

each R′ is independently selected from the group consisting of a hydrogen atom and an alkyl group having 1 to 4 carbon atoms;

each R₂ is independently a group having 0 to 10 carbon atoms and optionally containing a heteroatom;

each m2 is independently an integer of 0 to 3;

each m2′ is independently an integer of 0 to 4;

n is an integer of 0 to 4; and

a plurality of groups represented by:

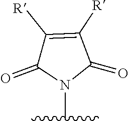

comprise at least a group of formula (0A) and a group of formula (0B).)
[7]

The film forming material for lithography according to [1] or [2], wherein the compound is represented by formula (3A).

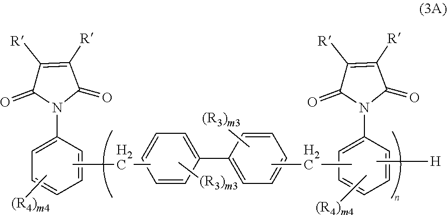

(In formula (3A),

each R′ is independently selected from the group consisting of a hydrogen atom and an alkyl group having 1 to 4 carbon atoms;

R₃ and R₄ are each independently a group having 0 to 10 carbon atoms and optionally containing a heteroatom;

each m3 is independently an integer of 0 to 4;

each m4 is independently an integer of 0 to 4;

n is an integer of 1 to 4; and

a plurality of groups represented by:

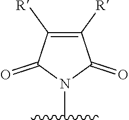

comprise at least a group of formula (0A) and a group of formula (0B).)
[8]

The film forming material for lithography according to any of [2] to [5], wherein the heteroatom is selected from the group consisting of oxygen, fluorine, and silicon.
[9]

The film forming material for lithography according to any of [1] to [8], further comprising a crosslinking agent.
[10]

The film forming material for lithography according to [9], wherein the crosslinking agent is at least one selected from the group consisting of a phenol compound, an epoxy compound, a cyanate compound, an amino compound, a benzoxazine compound, a melamine compound, a guanamine compound, a glycoluril compound, a urea compound, an isocyanate compound, and an azide compound.
[11]

The film forming material for lithography according to [9] or [10], wherein the crosslinking agent has at least one allyl group.
[12]

The film forming material for lithography according to any of [9] to [11], wherein a content ratio of the crosslinking agent is 0.1 to 100 parts by mass based on 100 parts by mass of a mass of the compound.
[13]

The film forming material for lithography according to any of [1] to [12], further comprising a crosslinking promoting agent.
[14]

The film forming material for lithography according to [13], wherein the crosslinking promoting agent comprises at least one selected from the group consisting of an amine, an imidazole, an organic phosphine, and a Lewis acid.
[15]

The film forming material for lithography according to [13] or [14], wherein a content ratio of the crosslinking promoting agent is 0.1 to 5 parts by mass based on 100 parts by mass of a mass of the compound.
[16]

The film forming material for lithography according to any of [1] to [15], further comprising a radical polymerization initiator.
[17]

The film forming material for lithography according to [16], wherein the radical polymerization initiator comprises at least one selected from the group consisting of a ketone-based photopolymerization initiator, an organic peroxide-based polymerization initiator, and an azo-based polymerization initiator.
[18]

The film forming material for lithography according to [16] or [17], wherein a content ratio of the radical polymerization initiator is 0.05 to 25 parts by mass based on 100 parts by mass of a mass of the compound.
[19]

A composition for film formation for lithography comprising the film forming material for lithography according to any of [1] to [18] and a solvent.
[20]

The composition for film formation for lithography according to [19], further comprising an acid generating agent.
[21]

The composition for film formation for lithography according to [19] or [20], further comprising a basic compound.
[22]

The composition for film formation for lithography according to any of [19] to [21], wherein the film for lithography is an underlayer film for lithography.
[23]

An underlayer film for lithography formed by using the composition for film formation for lithography according to [22].
[24]

A method for forming a resist pattern, comprising the steps of:

forming an underlayer film on a substrate by using the composition for film formation for lithography according to [22];

forming at least one photoresist layer on the underlayer film; and

irradiating a predetermined region of the photoresist layer with radiation for development.
[25]

A method for forming a pattern, comprising the steps of:

forming an underlayer film on a substrate by using the composition for film formation for lithography according to [22];

forming an intermediate layer film on the underlayer film by using a resist intermediate layer film material containing a silicon atom;

forming at least one photoresist layer on the intermediate layer film;

irradiating a predetermined region of the photoresist layer with radiation for development, thereby forming a resist pattern;

etching the intermediate layer film with the resist pattern as a mask;

etching the underlayer film with the obtained intermediate layer film pattern as an etching mask; and

etching the substrate with the obtained underlayer film pattern as an etching mask, thereby forming a pattern on the substrate.
[26]

A purification method comprising the steps of:

obtaining an organic phase by dissolving the film forming material for lithography according to any of [1] to [18] in a solvent; and

extracting impurities in the film forming material for lithography by bringing the organic phase into contact with an acidic aqueous solution (a first extraction step),

wherein

the solvent used in the step of obtaining the organic phase contains a solvent that does not inadvertently mix with water.
[27]

The purification method according to [26], wherein:

the acidic aqueous solution is an aqueous mineral acid solution or an aqueous organic acid solution;

the aqueous mineral acid solution contains one or more selected from the group consisting of hydrochloric acid, sulfuric acid, nitric acid, and phosphoric acid; and

the aqueous organic acid solution contains one or more selected from the group consisting of acetic acid, propionic acid, oxalic acid, malonic acid, succinic acid, fumaric acid, maleic acid, tartaric acid, citric acid, methanesulfonic acid, phenolsulfonic acid, p-toluenesulfonic acid, and trifluoroacetic acid.
[28]

The purification method according to [26] or [27], wherein the solvent that does not inadvertently mix with water is one or more solvents selected from the group consisting of toluene, 2-heptanone, cyclohexanone, cyclopentanone, methyl isobutyl ketone, propylene glycol monomethyl ether acetate, and ethyl acetate.
[29]

The purification method according to any of [26] to [28], further comprising the step of extracting impurities in the film forming material for lithography by bringing the organic phase into contact with water after the first extraction step (a second extraction step).

### Advantageous Effects of Invention

The present invention can provide a film forming material for lithography that is applicable to a wet process, and is useful for forming a photoresist underlayer film excellent in heat resistance, etching resistance, embedding properties to a substrate having difference in level, and film flatness; a composition for film formation for lithography comprising the material; as well as an underlayer film for lithography and a method for forming a pattern by using the composition.

### Description of Embodiments

Hereinafter, embodiments of the present invention will be described. The embodiments described below are given merely for illustrating the present invention. The present invention is not limited only by these embodiments.

### [Film Forming Material for Lithography]

One of the present embodiments is:

a film forming material for lithography comprising a compound having:

a group of formula (0A):

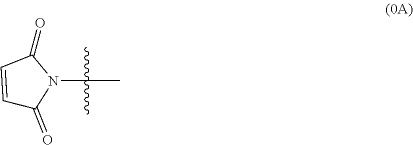 [SMILES: *N1C(=O)C=CC1=O]

and

a group of formula (0B):

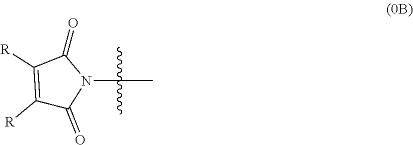

(In formula (0B),

each R is independently selected from the group consisting of a hydrogen atom and an alkyl group having 1 to 4 carbon atoms, provided that at least one R is an alkyl group having 1 to 4 carbon atoms.)

It is preferable that the compound having a group of formula (0A) and a group of formula (0B) (hereinafter, may be referred to as a “citramaleimide compound” in the present specification) have one or more groups of formula (0A) and one or more groups of formula (0B). The citramaleimide compound can be obtained by conducting a ring closure reaction with dehydration of, for example, a compound having one or more primary amino groups in the molecule, maleic anhydride, and citraconic anhydride. Examples of the citramaleimide compound may include, for example, a polycitramaleimide compound and a citramaleimide resin.

Also, the film forming material for lithography of the present invention only needs to comprise at least a compound having a group of formula (0A) and a group of formula (0B), and may also comprise another compound having a group of formula (0A) and/or compound having a group of formula (0B).

Examples of the compound having a group of formula (0A) include a compound having two groups of formula (0A) in the molecule, and examples of the compound having a group of formula (0B) include a compound having two groups of formula (0B) in the molecule.

From the viewpoint of heat resistance and etching resistance, the content of the citramaleimide compound in the film forming material for lithography of the present embodiment is preferably 51 to 100% by mass, more preferably 60 to 100% by mass, still more preferably 70 to 100% by mass, and even more preferably 80 to 100% by mass.

The citramaleimide compound in the film forming material for lithography of the present embodiment can also be used as an additive agent in order to improve the heat resistance of a conventional, underlayer film forming composition. In that case, the content of the citramaleimide compound is preferably 1 to 50% by mass and more preferably 1 to 30% by mass.

The citramaleimide compound in the film forming material for lithography of the present embodiment is characterized by having a function other than those as an acid generating agent for film formation for lithography or as a basic compound.

It is preferable that the molecular weight of the citramaleimide compound in the present embodiment be 450 or more. When the molecular weight is 450 or more, the production of sublimates or decomposition products tends to be suppressed even with high temperature baking during thin film formation. The molecular weight is more preferably 500 or more, still more preferably 550 or more, and even more preferably 600 or more. Although the upper limit of the molecular weight is not particularly limited, it may be, for example, 2000, 1750, 1500, 1250, 1000, or the like.

It is more preferable that the citramaleimide compound in the present embodiment be a compound represented by the following formula (LAfl:

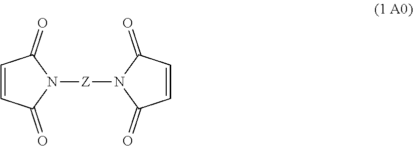

(In formula (1A₀),

each R is independently selected from the group consisting of a hydrogen atom and an alkyl group having 1 to 4 carbon atoms, provided that at least one R is an alkyl group having 1 to 4 carbon atoms; and

Z is a divalent hydrocarbon group having 1 to 100 carbon atoms and optionally containing a heteroatom.)

As mentioned above, the film forming material for lithography of the present invention may also comprise, in addition to the citramaleimide compound, another compound having a group of formula (0A) and/or compound having a group of formula (0B).

The compound having a group of formula (0A) and the compound having a group of formula (0B) are, for example, bismaleimide and biscitraconimide represented by the following structures, respectively.

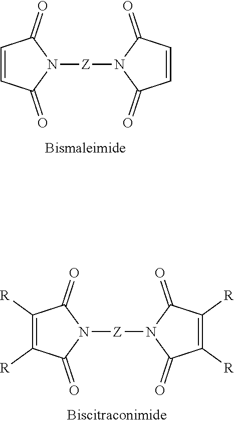

(In the above structures, Z is as defined in Z in formula (1A₀), and corresponds to a divalent hydrocarbon group moiety having 1 to 100 carbon atoms and optionally containing a heteroatom in formula (1A), which will be mentioned later.)

The number of carbon atoms in the hydrocarbon group may be 1 to 80, 1 to 60, 1 to 40, 1 to 20, or the like. Examples of the heteroatom may include oxygen, nitrogen, sulfur, fluorine, silicon, and the like, and among them, oxygen, fluorine, and silicon are preferable.

It is more preferable that the citramaleimide compound in the present embodiment be a compound represented by the following formula (1A):

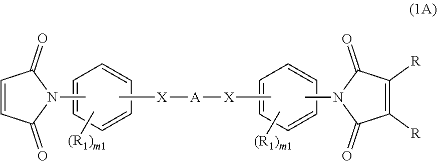

In formula (1A),

each R is independently selected from the group consisting of a hydrogen atom and an alkyl group having 1 to 4 carbon atoms, provided that at least one R is an alkyl group having 1 to 4 carbon atoms;

each X is independently selected from the group consisting of a single bond, —O—, —CH₂—, —C(CH₃)₂—, —CO—, —C(CF₃)₂—, —CONH—, and —COO—;

A is selected from the group consisting of a single bond, an oxygen atom, and a divalent hydrocarbon group having 1 to 80 carbon atoms and optionally containing a heteroatom (for example, oxygen, nitrogen, sulfur, or fluorine);

each R₁ is independently a group having 0 to 30 carbon atoms and optionally containing a heteroatom (for example, oxygen, nitrogen, sulfur, fluorine, chlorine, bromine, or iodine); and

each m1 is independently an integer of 0 to 4.

More preferably, from the viewpoint of improvement in heat resistance, in formula (1A), A is a single bond, an oxygen atom, —(CH₂)ₚ—, —CH₂C (CH₃)₂CH₂—, —(C(CH₃)₂)ₚ—, —(O(CH₂)_{q})ₚ—, —(O(C₆H₄))ₚ—, or any of the following structures:

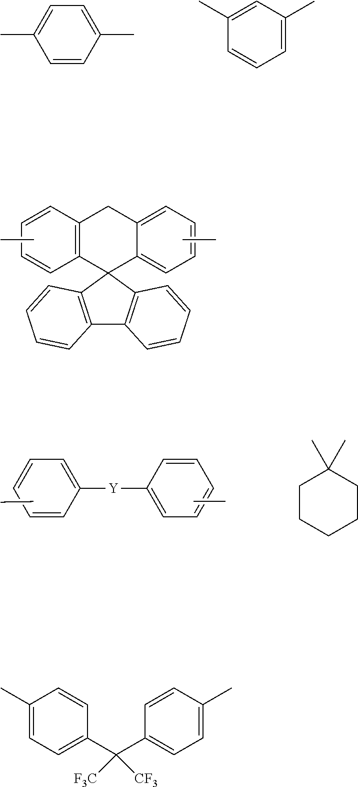

Y is a single bond, —O—, —CH₂—, —C(CH₂—, —C(CH₃)₂—, —C(CF₃)₂—,

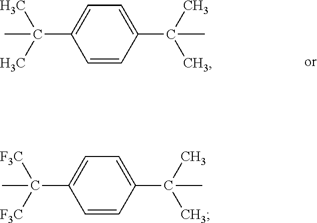

p is an integer of 0 to 20; and

q is an integer of 0 to 4.

Still more preferably, in formula (1A),

each X is independently a single bond, —O—, —C(CH₃)₂—, —CO—, or —COO—;

A is a single bond, an oxygen atom, or the following structures:

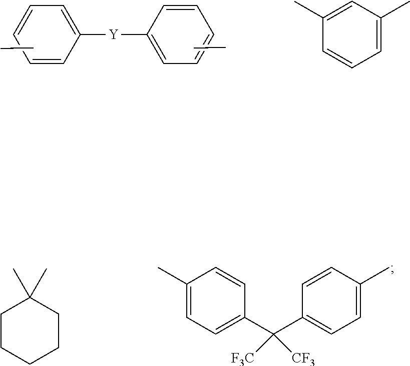

and

Y is —C(CH₃)₂— or —C(CF₃)₂—.

X is preferably a single bond from the viewpoint of heat resistance, and is preferably —COO— from the viewpoint of solubility.

Y is preferably a single bond from the viewpoint of improvement in heat resistance.

R₁ is preferably a group having 0 to 20 or 0 to 10 carbon atoms and optionally containing a heteroatom (for example, oxygen, nitrogen, sulfur, fluorine, chlorine, bromine, or iodine). R₁ is preferably a hydrocarbon group from the viewpoint of improvement in solubility in an organic solvent. For example, examples of R₁ include an alkyl group (for example, an alkyl group having 1 to 6 or 1 to 3 carbon atoms) and the like, and specific examples thereof include a methyl group, an ethyl group, and the like.

m1 is preferably an integer of 0 to 2, and is more preferably 1 or 2 from the viewpoint of the availability of raw materials and improvement in solubility.

From the viewpoint of improvement in heat resistance, it is preferable that the citramaleimide compound in the present embodiment be a compound represented by the following formula (2A) or the following formula (3A). Also, it is more preferable that the compound represented by the following formula (2A) or the following formula (3A) have at least one group of the following formula (0B′):

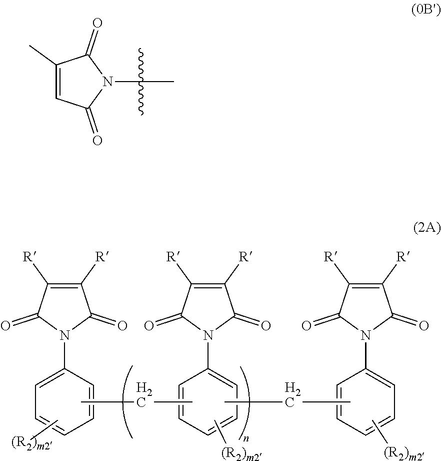

In the above formula (2),

each R′ is independently selected from the group consisting of a hydrogen atom and an alkyl group having 1 to 4 carbon atoms; and

each R₂ is independently a group having 0 to 10 carbon atoms and optionally containing a heteroatom (for example, oxygen, nitrogen, sulfur, fluorine, chlorine, bromine, or iodine). In addition, R₂ is preferably a hydrocarbon group from the viewpoint of improvement in solubility in an organic solvent. For example, examples of R₂ include an alkyl group (for example, an alkyl group having 1 to 6 or 1 to 3 carbon atoms) and the like, and specific examples thereof include a methyl group, an ethyl group, and the like.

Each m2 is independently an integer of 0 to 3. In addition, m2 is preferably 0 or 1, and is more preferably 0 from the viewpoint of the availability of raw materials.

Each m2′ is independently an integer of 0 to 4. In addition, m2′ is preferably 0 or 1, and is more preferably 0 from the viewpoint of the availability of raw materials.

n is an integer of 0 to 4. In addition, n is preferably an integer of 1 to 4, and is more preferably an integer of 1 to 3 from the viewpoint of improvement in heat resistance.

In addition, when n is 1 or more, monomers that may cause sublimates are removed, and compatibility of flatness and heat resistance can be expected, and it is more preferable that n be 1.

A plurality of groups represented by:

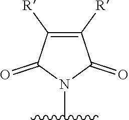

comprise at least a group of formula (0A) and a group of formula (0B).

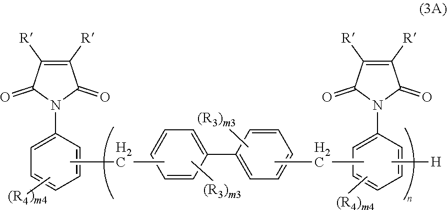

In the above formula (3A),

each R′ is independently selected from the group consisting of a hydrogen atom and an alkyl group having 1 to 4 carbon atoms; and

R₃ and R₄ are each independently a group having 0 to 10 carbon atoms and optionally containing a heteroatom (for example, oxygen, nitrogen, sulfur, fluorine, chlorine, bromine, or iodine). In addition, R₃ and R₄ are preferably hydrocarbon groups from the viewpoint of improvement in solubility in an organic solvent. For example, examples of R₃ and R₄ include an alkyl group (for example, an alkyl group having 1 to 6 or 1 to 3 carbon atoms) and the like, and specific examples thereof include a methyl group, an ethyl group, and the like.

Each m3 is independently an integer of 0 to 4. In addition, m3 is preferably an integer of 0 to 2, and is more preferably 0 from the viewpoint of the availability of raw materials.

Each m4 is independently an integer of 0 to 4. In addition, m4 is preferably an integer of 0 to 2, and is more preferably 0 from the viewpoint of the availability of raw materials.

n is an integer of 1 to 4. In addition, n is preferably an integer of 1 to 2 from the viewpoint of the availability of raw materials. Furthermore, n is preferably an integer of 2 to 4 from the viewpoint of improvement in heat resistance.

In addition, when n is 2 or more, monomers that may cause sublimates are removed, and compatibility of flatness and heat resistance can be expected, and it is more preferable that n be 2.

The film forming material for lithography of the present embodiment is applicable to a wet process. In addition, the film forming material for lithography of the present embodiment has an aromatic structure and also has a rigid maleimide skeleton and citraconimide skeleton, and therefore, when it is baked at a high temperature, its maleimide group and citraconimide group undergo a crosslinking reaction even on its own, thereby expressing high heat resistance. As a result, deterioration of the film upon baking at a high temperature is suppressed and an underlayer film excellent in etching resistance to oxygen plasma etching and the like can be formed. Furthermore, even though the film forming material for lithography of the present embodiment has an aromatic structure, its solubility in an organic solvent is high and its solubility in a safe solvent is high.

Furthermore, an underlayer film for lithography composed of the composition for film formation for lithography of the present embodiment, which will be mentioned later, is not only excellent in embedding properties to a substrate having difference in level and film flatness, thereby having a good stability of the product quality, but also excellent in adhesiveness to a resist layer or a resist intermediate layer film material, and thus, an excellent resist pattern can be obtained.

Specific examples of the citramaleimide compound to be used in the present embodiment include: citramaleimides obtained from phenylene skeleton-containing bisamines such as m-phenylenediamine, 4-methyl-1,3-phenylenediamine, 4,4-diaminodiphenylmethane, 4,4-diaminodiphenylsulfone, 1,3-bis(3-aminophenoxy)benzene, 1,3-bis(4-aminophenoxy)benzene, 1,4-bis(3-aminophenoxy)benzene, and 1,4-bis(4-aminophenoxy)benzene;

citramaleimides obtained from diphenylalkane skeleton-containing bisamines such as bis(3-ethyl-5-methyl-4-aminophenyl)methane, 1,1-bis(3-ethyl-5-methyl-4-aminophenyl)ethane, 2,2-bis(3-ethyl-5-methyl-4-aminophenyl)propane, N,N′-4,4′-diamino 3,3′-dimethyl-diphenylmethane, N,N′-4,4′-diamino 3,3′-dimethyl-1,1-diphenylethane, N,N′-4,4′-diamino 3,3′-dimethyl-1,1-diphenylpropane, N,N′-4,4′-diamino 3,3′-diethyl-diphenylmethane, N,N′-4,4′-diamino 3,3′-di-n-propyl-diphenylmethane, and N,N′-4,4′-diamino 3,3′-di-n-butyl-diphenylmethane;

citramaleimides obtained from biphenyl skeleton-containing bisamines such as N,N′-4,4′-diamino 3,3′-dimethyl-biphenylene and N,N′-4,4′-diamino 3,3′-diethyl-biphenylene;

citramaleimides obtained from aliphatic skeleton bisamines such as 1,6-hexanediamine, 1,6-bisamino(2,2,4-trimethyl) hexane, 1,3-dimethylenecyclohexanediamine, and 1,4-dimethylenecyclohexanediamine;

citramaleimides obtained from diamino siloxanes such as 1,3-bis(3-aminopropyl)-1,1,2,2-tetramethyl disiloxane, 1,3-bis(3-aminobutyl)-1,1,2,2-tetramethyl disiloxane, bis(4-aminophenoxy)dimethyl silane, 1,3-bis(4-aminophenoxy)tetramethyl disiloxane, 1, 1,3,3-tetramethyl-1,3-bis(4-aminophenyl)disiloxane, 1, 1,3,3-tetraphenoxy-1,3-bis(2-aminoethyl)disiloxane, 1,1,3,3-tetraphenyl-1,3-bis(2-aminoethyl)disiloxane, 1,1,3,3-tetraphenyl-1,3-bis(3-aminopropyl)disiloxane, 1,1,3,3-tetramethyl-1,3-bis(2-aminoethyl)disiloxane, 1,1,3,3-tetramethyl-1,3-bis(3-aminopropyl)disiloxane, 1,1,3,3-tetramethyl-1,3-bis(4-aminobutyl)disiloxane, 1,3-dimethyl-1,3-dimethoxy-1,3-bis(4-aminobutyl)disiloxane, 1,1,3,3,5,5-hexamethyl-1,5-bis(4-aminophenyl)trisiloxane, 1,1,5,5-tetraphenyl-3,3-dimethyl-1,5-bis(3-aminopropyl)trisiloxane, 1,1,5,5-tetraphenyl-3,3-dimethoxy-1,5-bis(4-aminobutyl)trisiloxane, 1,1,5,5-tetraphenyl-3,3-dimethoxy-1,5-bis(5-aminopentyl)trisiloxane, 1,1,5,5-tetramethyl-3,3-dimethoxy-1,5-bis(2-aminoethyl)trisiloxane, 1,1,5,5-tetramethyl-3,3-dimethoxy-1,5-bis(4-aminobutyl)trisiloxane, 1,1,5,5-tetramethyl-3,3-dimethoxy-1,5-bis(5-aminopentyl)trisiloxane, 1, 1,3,3,5,5-hexamethyl-1,5-bis(3-aminopropyl)trisiloxane, 1, 1,3,3,5,5-hexaethyl-1,5-bis(3-aminopropyl)trisiloxane, and 1,1,3,3,5,5-hexapropyl-1,5-bis(3-aminopropyl)trisiloxane; and the like.

Among the citramaleimide compounds described above, bis(3-ethyl-5-methyl-4-maleimidephenyl)methane, N,N′-4,4′-[3,3′-dimethyl-diphenylmethane]citraconimide maleimide, and N,N′-4,4′-[3,3′-diethyldiphenylmethane]citraconimide maleimide are particularly preferable because they are excellent in curability, as well as heat resistance.

Among the biscitraconimide compounds described above, bis(3-ethyl-5-methyl-4-citraconimidephenyl)methane, N,N′-4,4′-[3,3′-dimethyl-diphenylmethane]citraconimide maleimide, and N,NY-4,4′-[3,3′-diethyldiphenylmethane]citraconimide maleimide are particularly preferable because they are excellent in solvent solubility.

### <Crosslinking Agent>

The film forming material for lithography of the present embodiment may comprise a crosslinking agent, if required, in addition to the compound having a group of formula (0A) and a group of formula (0B), from the viewpoint of lowering the curing temperature, suppressing intermixing, and the like.

The crosslinking agent is not particularly limited as long as it undergoes a crosslinking reaction with a maleimide group and a citraconimide group, and any of publicly known crosslinking systems can be applied. Examples of the crosslinking agent that can be used in the present embodiment include, but are not particularly limited to, a phenol compound, an epoxy compound, a cyanate compound, an amino compound, a benzoxazine compound, an acrylate compound, a melamine compound, a guanamine compound, a glycoluril compound, a urea compound, an isocyanate compound, an azide compound, and the like. These crosslinking agents can be used alone as one kind, or can be used in combination of two or more kinds. Among them, a benzoxazine compound, an epoxy compound, or a cyanate compound is preferable, and a benzoxazine compound is more preferable from the viewpoint of improvement in etching resistance.

In a crosslinking reaction of a maleimide group, a citraconimide group, and a crosslinking agent, for example, an active group these crosslinking agents have (a phenolic hydroxy group, an epoxy group, a cyanate group, an amino group, or a phenolic hydroxy group formed by ring opening of the alicyclic site of benzoxazine) undergoes an addition reaction with a carbon-carbon double bond that constitutes a maleimide group and a citraconimide group to form crosslinkage. Besides, two carbon-carbon double bonds that the compound in the present embodiment has are polymerized to form crosslinkage.

As the above phenol compound, a publicly known compound can be used. For example, examples thereof include those described in International Publication No. WO 2018-016614. Preferably, an aralkyl-based phenolic resin is desirable from the viewpoint of heat resistance and solubility.

As the above epoxy compound, a publicly known compound can be used and is selected from among compounds having two or more epoxy groups in one molecule. For example, examples thereof include those described in International Publication No. WO 2018/016614. These epoxy resins may be used alone, or may be used in combination of two or more kinds. An epoxy resin that is in a solid state at normal temperature, such as an epoxy resin obtained from a phenol aralkyl resin or a biphenyl aralkyl resin is preferable from the viewpoint of heat resistance and solubility.

The above cyanate compound is not particularly limited as long as the compound has two or more cyanate groups in one molecule, and a publicly known compound can be used. For example, examples thereof include those described in International Publication No. WO 2011-108524, but preferable examples of the cyanate compound in the present embodiment include cyanate compounds having a structure where hydroxy groups of a compound having two or more hydroxy groups in one molecule are substituted with cyanate groups. Also, the cyanate compound preferably has an aromatic group, and those having a structure in which a cyanate group is directly bonded to the aromatic group can be suitably used. Examples of such a cyanate compound include those described in International Publication No. WO 2018-016614. These cyanate compounds may be used alone, or may be used in arbitrary combination of two or more kinds. Also, the above cyanate compound may be in any form of a monomer, an oligomer, and a resin.

Examples of the above amino compound include those described in International Publication No. WO 2018-016614.

The structure of oxazine of the above benzoxazine compound is not particularly limited, and examples thereof include a structure of oxazine having an aromatic group including a condensed polycyclic aromatic group, such as benzoxazine and naphthoxazine.

Examples of the benzoxazine compound include, for example, compounds represented by the following general formulas (a) to (f). Note that, in the general formulas described below, a bond displayed toward the center of a ring indicates a bond to any carbon that constitutes the ring and to which a substituent can be bonded.

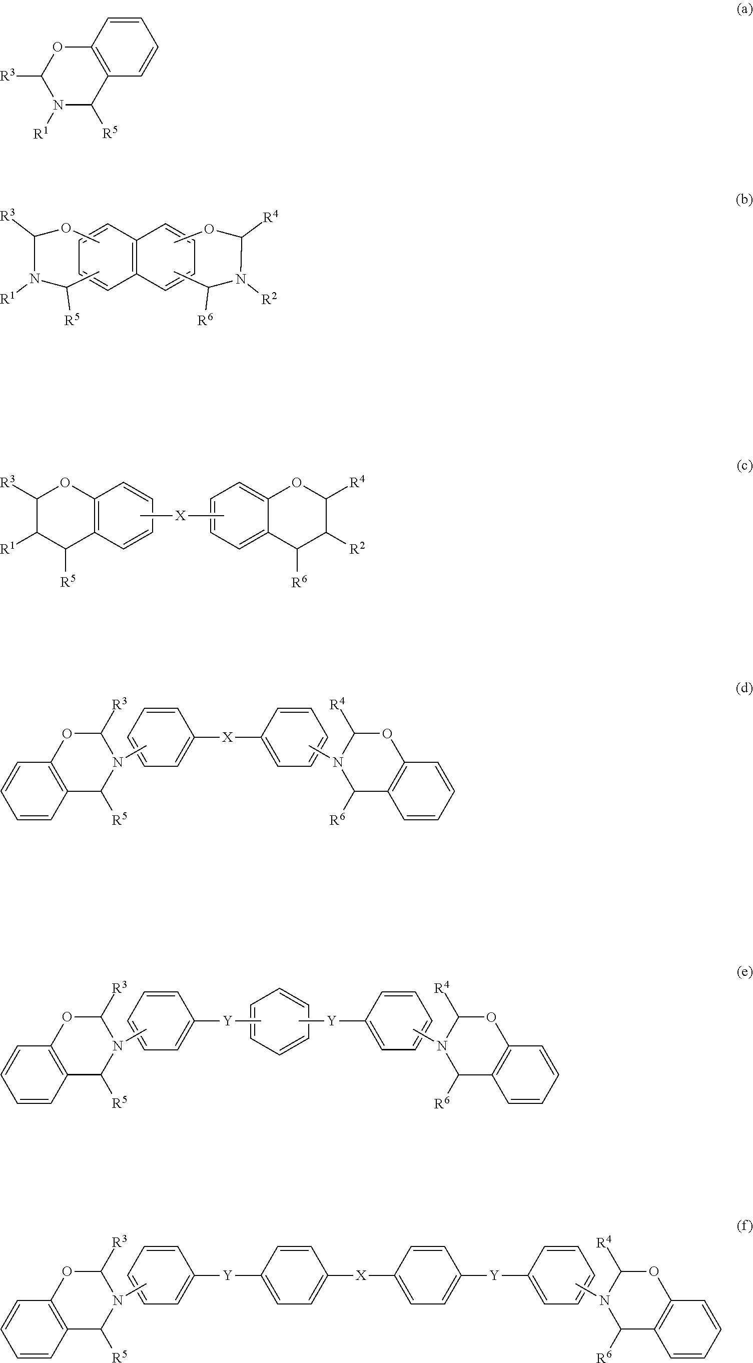

In the general formulas (a) to (c), R¹ and R² each independently represent an organic group having 1 to 30 carbon atoms. In addition, in the general formulas (a) to (f), R³ to R⁶ each independently represent hydrogen or a hydrocarbon group having 1 to 6 carbon atoms. Moreover, in the above general formulas (c), (d), and (f), X independently represents a single bond, —O—, —S—, —S—S—, —SO₂—, —CO—, —CONH—, —NHCO—, —C(CH₃)₂—, —C(CF₃)₂—, —(CH₂)m-, —O—(CH₂)m-O—, or —S—(CH₂)m-S—. Here, m is an integer of 1 to 6. In addition, in the general formulas (e) and (f), Y independently represents a single bond, —O—, —S—, —CO—, —C(CH₃)₂—, —C(CF₃)₂—, or alkylene having 1 to 3 carbon atoms.

Moreover, the benzoxazine compound includes an oligomer or polymer having an oxazine structure as a side chain, and an oligomer or polymer having a benzoxazine structure in the main chain.

The benzoxazine compound can be produced in a similar method as a method described in International Publication No. WO 2004/009708, Japanese Patent Application Laid-Open No. 11-12258, or Japanese Patent Application Laid-Open No. 2004-352670.

Examples of the above melamine compound include those described in International Publication No. WO 2018-016614.

Examples of the above guanamine compound include those described in International Publication No. WO 2018-016614.

Examples of the above glycoluril compound include those described in International Publication No. WO 2018-016614.

Examples of the above urea compound include those described in International Publication No. WO 2018-016614.

In the present embodiment, a crosslinking agent having at least one allyl group may be used from the viewpoint of improvement in crosslinkability. Specific examples of the crosslinking agent having at least one allyl group include, but are not limited to, those described in International Publication No. WO 2018-016614. These crosslinking agents having at least one allyl group may be alone, or may be a mixture of two or more kinds. Among them, an allylphenol such as 2,2-bis(3-allyl-4-hydroxyphenyl)propane, 1,1,1,3,3,3-hexafluoro-2,2-bis(3-allyl-4-hydroxyphenyl)propane, bis(3-allyl-4-hydroxyphenyl)sulfone, bis(3-allyl-4-hydroxyphenyl)sulfide, and bis(3-allyl-4-hydroxyphenyl) ether is preferable from the viewpoint of excellent compatibility with the compound having a group of 0A and a group of 0B.

The film for lithography of the present embodiment can be formed by crosslinking and curing the film forming material for lithography of the present embodiment alone, or after compounding with the above crosslinking agent, by a publicly known method. Examples of the crosslinking method include approaches such as heat curing and light curing.

The content ratio of the crosslinking agent is normally in the range of 0.1 to 10000 parts by mass based on 100 parts by mass of the mass of the above citramaleimide compound, preferably in the range of 0.1 to 1000 parts by mass from the viewpoint of heat resistance and solubility, more preferably in the range of 0.1 to 100 parts by mass, still more preferably in the range of 1 to 50 parts by mass, and even more preferably in the range of 1 to 30 parts by mass.

In the film forming material for lithography of the present embodiment, if required, a crosslinking promoting agent for accelerating crosslinking reaction and curing reaction can be used.

The above crosslinking promoting agent is not particularly limited as long as it accelerates crosslinking or curing reaction, and examples thereof include an amine, an imidazole, an organic phosphine, and a Lewis acid. These crosslinking promoting agents can be used alone as one kind, or can be used in combination of two or more kinds. Among them, an imidazole or an organic phosphine is preferable, and an imidazole is more preferable from the viewpoint of decrease in crosslinking temperature.

Examples of the above crosslinking promoting agent include, for example, those described in International Publication No. WO 2018-016614.

The amount of the crosslinking promoting agent to be compounded is normally preferably in the range of 0.01 to 10 parts by mass based on 100 parts by mass of the mass of the compound having a group of formula (0A) and a group of formula (0B), and is more preferably in the range of 0.01 to 5 parts by mass and still more preferably in the range of 0.01 to 3 parts by mass, from the viewpoint of easy control and cost efficiency.

In the film forming material for lithography of the present embodiment, if required, a latent base generating agent for accelerating crosslinking reaction and curing reaction can be used. The latent base generating agent is a curing accelerator that exhibits no activity under normal storage conditions, but is active in response to external stimuli (for example, heat, light, or the like). As the base generating agent, those generating a base by thermal decomposition, those generating a base by light irradiation (photobase generating agents), and the like are known, any of which can be used.

The photobase generating agent is a neutral compound that produces a base by exposing the photobase generating agent to electromagnetic wave. Examples of those generating an amine include, for example, a benzyl carbamate, a benzoyl carbamate, an O-carbamoylhydroxyamine, and an O-carbamoyloxime, as well as RR′—N—CO—OR″ (where R and R′ are each independently hydrogen or a lower alkyl group; and R″ is nitrobenzyl or α-methyl-nitrobenzyl). Particularly, in order to ensure storage stability upon adding the photobase generating agent to the solution and inhibiting volatilization upon baking due to low vapor pressure, preferable is a borate compound generating a tertiary amine, a quaternary ammonium salt comprising a dithiocarbamate as an anion (C. E. Hoyle, et. al., Macromolecules, 32, 2793 (1999)), or the like.

Specific examples of the above latent base generating agent may include, for example, those described below; however, the present invention is not limited in any way by them.

(Examples of hexaammineruthenium(III) triphenylalkylborate)

Hexaammineruthenium(III) tris(triphenylmethylborate), hexaammineruthenium(III) tris(triphenylethylborate), hexaammineruthenium(III) tris(triphenylpropylborate), hexaammineruthenium(III) tris(triphenylbutylborate), hexaammineruthenium(III) tris(triphenylhexylborate), hexaammineruthenium(III) tris(triphenyloctylborate), hexaammineruthenium(III) tris(triphenyloctadecylborate), hexaammineruthenium(III) tris(triphenylisopropylborate), hexaammineruthenium(III) tris(triphenylisobutylborate), hexaammineruthenium(III) tris(triphenyl-sec-butylborate), hexaammineruthenium(III) tris(triphenyl-tert-butylborate), hexaammineruthenium(III) tris(triphenylneopentylborate), and the like.

(Examples of hexaammineruthenium(III) triphenylborate)

Hexaammineruthenium(III) tris(triphenylcyclopentylborate), hexaammineruthenium(III) tris(triphenylcyclohexylborate), hexaammineruthenium(III) tris[triphenyl(4-decylcyclohexyl)borate], hexaammineruthenium(III) tris[triphenyl(fluoromethyl)borate], hexaammineruthenium(III) tris[triphenyl(chloromethyl)borate], hexaammineruthenium(III) tris[triphenyl(bromomethyl)borate], hexaammineruthenium(III) tris[triphenyl(trifluoromethyl)borate], hexaammineruthenium(III) tris[triphenyl(trichloromethyl)borate], hexaammineruthenium(III) tris[triphenyl(hydroxymethyl)borate], hexaammineruthenium(III) tris[triphenyl(carboxymethyl)borate], hexaammineruthenium(III) tris[triphenyl(cyanomethyl)borate], hexaammineruthenium(III) tris[triphenyl(nitromethyl)borate], hexaammineruthenium(III) tris[triphenyl(azidomethyl)borate], and the like.

(Examples of hexaammineruthenium(III) triarylbutylborate)

Hexaammineruthenium(III) tris[tris(1-naphthyl)butylborate], hexaammineruthenium(III) tris[tris(2-naphthyl)butylborate], hexaammineruthenium(III) tris[tris(o-tolyl)butylborate], hexaammineruthenium(III) tris[tris(m-tolyl)butylborate], hexaammineruthenium(III) tris[tris(p-tolyl)butylborate], hexaammineruthenium(III) tris[tris(2,3-xylyl)butylborate], hexaammineruthenium(III) tris[tris(2,5-xylyl)butylborate], and the like.

(Examples of ruthenium(III) tris(triphenylbutylborate))

Tris(ethylenediamine)ruthenium(III) tris(triphenylbutylborate), cis-diamminebis(ethylenediamine)ruthenium(III) tris(triphenylbutylborate), trans-diamminebis(ethylenediamine)ruthenium(III) tris(triphenylbutylborate), tris(trimethylenediamine)ruthenium(III) tris(triphenylbutylborate), tris(propylenediamine)ruthenium(III) tris(triphenylbutylborate), tetraammine{(−) (propylenediamine)}ruthenium(III) tris(triphenylbutylborate), tris(trans-1,2-cyclohexanediamine)ruthenium(III) tris(triphenylbutylborate), bis(diethylenetriamine)ruthenium(III) tris(triphenylbutylborate), bis(pyridine)bis(ethylenediamine)ruthenium(III) tris(triphenylbutylborate), bis(imidazole)bis(ethylenediamine)ruthenium(III) tris(triphenylbutylborate), and the like.

The above latent base generating agent can be readily produced by mixing a halide salt, sulfate salt, nitrate salt, acetate salt, or the like of each complex ion with an alkali metal borate salt in an appropriate solvent such as water, an alcohol, or a water-containing organic solvent. These halide salt, sulfate salt, nitrate salt, acetate salt, and the like of each complex ion, which are raw materials, are easily available as commercial products. Besides, the synthesis method therefor is described in, for example, New Experimental Chemistry Lecture Vol. 8 (Synthesis of Inorganic Compounds III), edited by The Chemical Society of Japan, Maruzen Co., Ltd., 1977, and the like.

The content of the latent base generating agent may be any amount as long as it is a stoichiometrically required amount relative to the mass of the above maleimide compound, but it is preferably 0.01 to 25 parts by mass and more preferably 0.01 to 10 parts by mass, based on 100 parts by mass of the mass of the above maleimide compound. When the content of the latent base generating agent is 0.01 parts by mass or more, there is a tendency that curing of the maleimide compound can be prevented from being insufficient. On the other hand, when the content of the latent base generating agent is 25 parts by mass or less, there is a tendency that the long term storage stability of the film forming material for lithography at room temperature can be prevented from being impaired.

### <Radical Polymerization Initiator>

The film forming material for lithography of the present embodiment can contain, if required, a radical polymerization initiator. The radical polymerization initiator may be a photopolymerization initiator that initiates radical polymerization by light, or may be a thermal polymerization initiator that initiates radical polymerization by heat.

Examples of such a radical polymerization initiator include, for example, those described in International Publication No. WO 2018-016614. Examples of the radical polymerization initiator include, for example, a ketone-based photopolymerization initiator, an organic peroxide-based polymerization initiator, and an azo-based polymerization initiator. As the radical polymerization initiator according to the present embodiment, one kind thereof may be used alone, or two or more kinds thereof may be used in combination.

The content of the above radical polymerization initiator may be any amount as long as it is a stoichiometrically required amount relative to the mass of the above citramaleimide compound, but it is preferably 0.05 to 25 parts by mass and more preferably 0.1 to 10 parts by mass, based on 100 parts by mass of the mass of the above maleimide compound. When the content of the radical polymerization initiator is 0.05 parts by mass or more, there is a tendency that curing of the maleimide compound can be prevented from being insufficient. On the other hand, when the content of the radical polymerization initiator is 25 parts by mass or less, there is a tendency that the long term storage stability of the film forming material for lithography at room temperature can be prevented from being impaired.

### [Method for Purifying Film Forming Material for Lithography]

The film forming material for lithography of the present embodiment can be purified by washing with an acidic aqueous solution. The above purification method comprises a step in which the film forming material for lithography is dissolved in an organic solvent that does not inadvertently mix with water to obtain an organic phase, the organic phase is brought into contact with an acidic aqueous solution to carry out extraction treatment (a first extraction step), thereby transferring metals contained in the organic phase containing the film forming material for lithography and the organic solvent to an aqueous phase, and then, the organic phase and the aqueous phase are separated. According to the purification, the contents of various metals in the film forming material for lithography of the present embodiment can be reduced remarkably.

The organic solvent that does not inadvertently mix with water is not particularly limited, but is preferably an organic solvent that is safely applicable to semiconductor manufacturing processes. Normally, the amount of the organic solvent used is approximately 1 to 100 times by mass relative to the compound used.

Specific examples of the organic solvent to be used include, for example, those described in International Publication No. WO 2015/080240. Among them, toluene, 2-heptanone, cyclohexanone, cyclopentanone, methyl isobutyl ketone, propylene glycol monomethyl ether acetate, ethyl acetate, and the like are preferable, and cyclohexanone and propylene glycol monomethyl ether acetate are more preferable. These organic solvents can be each used alone, or can also be used as a mixture of two or more kinds.

The above acidic aqueous solution is appropriately selected from aqueous solutions in which generally known organic or inorganic compounds are dissolved in water, and examples thereof include, for example, those described in International Publication No. WO 2015/080240. These acidic aqueous solutions can be each used alone, or can also be used as a combination of two or more kinds. Examples of the acidic aqueous solution may include, for example, an aqueous mineral acid solution and an aqueous organic acid solution. Examples of the aqueous mineral acid solution may include, for example, an aqueous solution comprising one or more selected from the group consisting of hydrochloric acid, sulfuric acid, nitric acid, and phosphoric acid. Examples of the aqueous organic acid solution may include, for example, an aqueous solution comprising one or more selected from the group consisting of acetic acid, propionic acid, oxalic acid, malonic acid, succinic acid, fumaric acid, maleic acid, tartaric acid, citric acid, methanesulfonic acid, phenolsulfonic acid, p-toluenesulfonic acid, and trifluoroacetic acid. Moreover, as the acidic aqueous solution, aqueous solutions of sulfuric acid, nitric acid, and a carboxylic acid such as acetic acid, oxalic acid, tartaric acid, and citric acid are preferable, aqueous solutions of sulfuric acid, oxalic acid, tartaric acid, and citric acid are more preferable, and an aqueous solution of oxalic acid is still more preferable. It is considered that a polyvalent carboxylic acid such as oxalic acid, tartaric acid, and citric acid coordinates with metal ions and provides a chelating effect, and thus is capable of removing more metals. In addition, as the water used herein, water, the metal content of which is small, such as ion exchanged water, is preferable according to the purpose of the present invention.

The pH of the above acidic aqueous solution is not particularly limited, but when the acidity of the aqueous solution is too high, it may have a negative influence on the used compound or resin. Therefore, the pH range is normally about 0 to 5, and is more preferably about pH 0 to 3.

The amount of the acidic aqueous solution used is not particularly limited, but when the amount is too small, it is required to increase the number of extraction treatments for removing metals, and on the other hand, when the amount of the aqueous solution is too large, the entire fluid volume becomes large, which may cause operational problems. Normally, the amount of the aqueous solution used is 10 to 200 parts by mass and preferably 20 to 100 parts by mass relative to the solution of the film forming material for lithography.

By bringing the acidic aqueous solution into contact with a solution (B) containing the film forming material for lithography and the organic solvent that does not inadvertently mix with water, metals can be extracted.

The temperature at which the above extraction treatment is carried out is generally in the range of 20 to 90° C., and preferably 30 to 80° C. The extraction operation is carried out, for example, by thoroughly mixing the solution (B) and the acidic aqueous solution by stirring or the like and then leaving the obtained mixed solution to stand still. Thereby, metals contained in the solution containing the used compound and the organic solvent are transferred to the aqueous phase. Also, by this operation, the acidity of the solution is lowered, and the deterioration of the used compound can be suppressed.

After the extraction treatment, the mixed solution is separated into a solution phase containing the used compound and the organic solvent and an aqueous phase, and the solution containing the organic solvent is recovered by decantation or the like. The time for leaving the mixed solution to stand still is not particularly limited, but when the time for leaving the mixed solution to stand still is too short, separation of the solution phase containing the organic solvent and the aqueous phase becomes poor, which is not preferable. Normally, the time for leaving the mixed solution to stand still is 1 minute or longer, more preferably 10 minutes or longer, and still more preferably 30 minutes or longer. In addition, while the extraction treatment may be carried out only once, it is also effective to repeat mixing, leaving-to-stand-still, and separating operations multiple times.

When such an extraction treatment is carried out using the acidic aqueous solution, after the treatment, it is preferable to further subject the recovered organic phase, which has been extracted from the aqueous solution and contains the organic solvent, to an extraction treatment with water (a second extraction step). The extraction operation is carried out by thoroughly mixing the organic phase and water by stirring or the like and then leaving the obtained mixed solution to stand still. The resultant mixed solution is separated into a solution phase containing the compound and the organic solvent and an aqueous phase, and thus the solution phase is recovered by decantation or the like. Water used herein is preferably water, the metal content of which is small, such as ion exchanged water, according to the purpose of the present invention. While the extraction treatment may be carried out only once, it is also effective to repeat mixing, leaving-to-stand-still, and separating operations multiple times. The proportions of both used in the extraction treatment and the temperature, time, and other conditions are not particularly limited, and may be the same as those of the previous contact treatment with the acidic aqueous solution.

Water that is unwantedly present in the thus-obtained solution containing the film forming material for lithography and the organic solvent can be easily removed by performing vacuum distillation or a like operation. Also, if required, the concentration of the compound can be regulated to be any concentration by adding an organic solvent.

A method for only obtaining the film forming material for lithography from the obtained solution containing the organic solvent can be carried out through a publicly known method such as reduced-pressure removal, separation by reprecipitation, and a combination thereof. Publicly known treatments such as concentration operation, filtration operation, centrifugation operation, and drying operation can be carried out if required.

### [Composition for Film Formation for Lithography]

A composition for film formation for lithography of the present embodiment comprises the above film forming material for lithography and a solvent. The film for lithography is, for example, an underlayer film for lithography.

The composition for film formation for lithography of the present embodiment can form a desired cured film by applying it on a base material, subsequently heating it to evaporate the solvent if necessary, and then heating or photoirradiating it. A method for applying the composition for film formation for lithography of the present embodiment is arbitrary, and a method such as spin coating, dipping, flow coating, inkjet coating, spraying, bar coating, gravure coating, slit coating, roll coating, transfer printing, brush coating, blade coating, and air knife coating can be employed appropriately.

The temperature at which the film is heated is not particularly limited according to the purpose of evaporating the solvent, and the heating can be carried out at, for example, 40 to 400° C. A method for heating is not particularly limited, and for example, the solvent may be evaporated under an appropriate atmosphere such as atmospheric air, an inert gas including nitrogen, and vacuum by using a hot plate or an oven. For the heating temperature and heating time, it is only required to select conditions suitable for a processing step for an electronic device that is aimed at and to select heating conditions by which physical property values of the obtained film satisfy requirements of the electronic device. Conditions for photoirradiation are not particularly limited, either, and it is only required to employ appropriate irradiation energy and irradiation time depending on a film forming material for lithography to be used.

### <Solvent>

A solvent to be used in the composition for film formation for lithography of the present embodiment is not particularly limited as long as it can at least dissolve the above citramaleimide compound, and any publicly known solvent can be used appropriately.

Specific examples of the solvent include, for example, those described in International Publication No. WO 2013/024779. These solvents can be used alone as one kind, or can be used in combination of two or more kinds.

Among the above solvents, cyclohexanone, propylene glycol monomethyl ether, propylene glycol monomethyl ether acetate, ethyl lactate, methyl hydroxyisobutyrate, or anisole is particularly preferable from the viewpoint of safety.

The content of the above solvent is not particularly limited and is preferably 25 to 9,900 parts by mass, more preferably 400 to 7,900 parts by mass, and still more preferably 900 to 4,900 parts by mass based on 100 parts by mass of the mass of the maleimide compound in the material for film formation for lithography, from the viewpoint of solubility and film formation.

### <Acid Generating Agent>

The composition for film formation for lithography of the present embodiment may contain an acid generating agent, if required, from the viewpoint of, for example, further accelerating crosslinking reaction. As the acid generating agent, those generating an acid by thermal decomposition, those generating an acid by light irradiation, and the like are known, any of which can be used.

Examples of the acid generating agent include, for example, those described in International Publication No. WO 2013/024779. Among them, in particular, onium salts such as triphenylsulfonium trifluoromethanesulfonate, (p-tert-butoxyphenyl) diphenylsulfonium trifluoromethanesulfonate, tris(p-tert-butoxyphenyl)sulfonium trifluoromethanesulfonate, triphenylsulfonium p-toluenesulfonate, (p-tert-butoxyphenyl) diphenylsulfonium p-toluenesulfonate, tris(p-tert-butoxyphenyl)sulfonium p-toluenesulfonate, trinaphthylsulfonium trifluoromethanesulfonate, cyclohexylmethyl(2-oxocyclohexyl)sulfonium trifluoromethanesulfonate, (2-norbornyl)methyl(2-oxocyclohexyl)sulfonium trifluoromethanesulfonate, and 1,2′-naphthylcarbonylmethyltetrahydrothiophenium triflate; diazomethane derivatives such as bis(benzenesulfonyl)diazomethane, bis(p-toluenesulfonyl)diazomethane, bis(cyclohexylsulfonyl)diazomethane, bis(n-butylsulfonyl)diazomethane, bis(isobutylsulfonyl)diazomethane, bis(sec-butylsulfonyl) diazomethane, bis(n-propylsulfonyl)diazomethane, bis(isopropylsulfonyl)diazomethane, and bis(tert-butylsulfonyl)diazomethane; glyoxime derivatives such as bis-(p-toluenesulfonyl)-α-dimethylglyoxime and bis-(n-butanesulfonyl)-α-dimethylglyoxime; bissulfone derivatives such as bisnaphthylsulfonylmethane; sulfonic acid ester derivatives of N-hydroxyimide compounds such as N-hydroxysuccinimide methanesulfonic acid ester, N-hydroxysuccinimide trifluoromethanesulfonic acid ester, N-hydroxysuccinimide 1-propanesulfonic acid ester, N-hydroxysuccinimide 2-propanesulfonic acid ester, N-hydroxysuccinimide 1-pentanesulfonic acid ester, N-hydroxysuccinimide p-toluenesulfonic acid ester, N-hydroxynaphthalimido methanesulfonic acid ester, and N-hydroxynaphthalimido benzenesulfonic acid ester are preferably used.

The content of the acid generating agent in the composition for film formation for lithography of the present embodiment is not particularly limited, and is preferably 0 to 50 parts by mass and more preferably 0 to 40 parts by mass based on 100 parts by mass of the mass of the maleimide compound in the film forming material for lithography. By setting the content of the acid generating agent to the preferable range mentioned above, crosslinking reaction tends to be enhanced. Also, a mixing event with a resist layer tends to be prevented.

### <Basic Compound>

The composition for underlayer film formation for lithography of the present embodiment may further contain a basic compound from the viewpoint of, for example, improving storage stability.

The above basic compound plays a role as a quencher against acids in order to prevent crosslinking reaction from proceeding due to a trace amount of an acid generated by the acid generating agent. Examples of such a basic compound include, but are not limited to, for example, primary, secondary or tertiary aliphatic amines, amine blends, aromatic amines, heterocyclic amines, nitrogen-containing compounds having a carboxy group, nitrogen-containing compounds having a sulfonyl group, nitrogen-containing compounds having a hydroxy group, nitrogen-containing compounds having a hydroxyphenyl group, alcoholic nitrogen-containing compounds, amide derivatives, and imide derivatives, described in International Publication No. WO 2013-024779.

The content of the basic compound in the composition for film formation for lithography of the present embodiment is not particularly limited, and is preferably 0 to 2 parts by mass and more preferably 0 to 1 part by mass based on 100 parts by mass of the mass of the maleimide compound in the film forming material for lithography. By setting the content of the basic compound to the preferable range mentioned above, storage stability tends to be enhanced without excessively deteriorating crosslinking reaction.

The composition for film formation for lithography of the present embodiment may further contain a publicly known additive agent. Examples of the publicly known additive agent include, but are not limited to, ultraviolet absorbers, antifoaming agents, colorants, pigments, nonionic surfactants, anionic surfactants, and cationic surfactants.

### [Underlayer Film for Lithography and Resist Pattern Formation Method]

The underlayer film for lithography of the present embodiment is formed by using the composition for film formation for lithography of the present embodiment.

A resist pattern formation method of the present embodiment comprises the steps of: forming an underlayer film on a substrate using the composition for film formation for lithography of the present embodiment (step (A-1)); forming at least one photoresist layer on the underlayer film (step (A-2)); and after the step (A-2), irradiating a predetermined region of the photoresist layer with radiation for development (step (A-3)).

Furthermore, one of the present embodiments is a pattern formation method, and such a pattern formation method comprises the steps of: forming an underlayer film on a substrate using the composition for film formation for lithography of the present embodiment (step (B-1)); forming an intermediate layer film on the underlayer film using a resist intermediate layer film material containing a silicon atom (step (B-2)); forming at least one photoresist layer on the intermediate layer film (step (B-3)); after the step (B-3), irradiating a predetermined region of the photoresist layer with radiation for development, thereby forming a resist pattern (step (B-4)); and after the step (B-4), etching the intermediate layer film with the resist pattern as a mask, etching the underlayer film with the obtained intermediate layer film pattern as an etching mask, and etching the substrate with the obtained underlayer film pattern as an etching mask, thereby forming a pattern on the substrate (step (B-5)).

The underlayer film for lithography of the present embodiment is not particularly limited by its formation method as long as it is formed from the composition for film formation for lithography of the present embodiment. A publicly known approach can be applied thereto. The underlayer film can be formed by, for example, applying the composition for film formation for lithography of the present embodiment onto a substrate by a publicly known coating method or printing method such as spin coating or screen printing, and then removing an organic solvent by volatilization or the like.

It is preferable to perform baking in the formation of the underlayer film, for preventing a mixing event with an upper layer resist while accelerating crosslinking reaction. In this case, the baking temperature is not particularly limited and is preferably in the range of 80 to 450° C., and more preferably 200 to 400° C. The baking time is not particularly limited and is preferably in the range of 10 to 300 seconds. The thickness of the underlayer film can be arbitrarily selected according to required performances and is not particularly limited, but is preferably 30 to 20,000 nm, more preferably 50 to 15,000 nm, and still more preferably 50 to 1000 nm.

After preparing the underlayer film on the substrate, in the case of a two-layer process, it is preferable to prepare a silicon-containing resist layer or a usual single-layer resist composed of hydrocarbon thereon, and in the case of a three-layer process, it is preferable to prepare a silicon-containing intermediate layer thereon and further prepare a single-layer resist layer not containing silicon thereon. In this case, for a photoresist material for forming this resist layer, a publicly known material can be used.

For the silicon-containing resist material for a two-layer process, a silicon atom-containing polymer such as a polysilsesquioxane derivative or a vinylsilane derivative is used as a base polymer, and a positive type photoresist material further containing an organic solvent, an acid generating agent, and if required, a basic compound or the like is preferably used, from the viewpoint of oxygen gas etching resistance. Here, a publicly known polymer that is used in this kind of resist material can be used as the silicon atom-containing polymer.

A polysilsesquioxane-based intermediate layer is preferably used as the silicon-containing intermediate layer for a three-layer process. By imparting effects as an antireflection film to the intermediate layer, there is a tendency that reflection can be effectively suppressed. For example, use of a material containing a large amount of an aromatic group and having high substrate etching resistance as the underlayer film in a process for exposure at 193 nm tends to increase a k value and enhance substrate reflection. However, the intermediate layer suppresses the reflection so that the substrate reflection can be 0.5% or less. The intermediate layer having such an antireflection effect is not limited, and polysilsesquioxane that crosslinks by an acid or heat in which a light absorbing group having a phenyl group or a silicon-silicon bond is introduced is preferably used for exposure at 193 nm.

Alternatively, an intermediate layer formed by chemical vapour deposition (CVD) may be used. The intermediate layer highly effective as an antireflection film prepared by CVD is not limited, and, for example, a SiON film is known. In general, the formation of an intermediate layer by a wet process such as spin coating or screen printing is more convenient and more advantageous in cost than CVD. The upper layer resist for a three-layer process may be positive type or negative type, and the same as a single-layer resist generally used can be used.

The underlayer film of the present embodiment can also be used as an antireflection film for usual single-layer resists or an underlying material for suppression of pattern collapse. The underlayer film of the present embodiment is excellent in etching resistance for an underlying process and can be expected to also function as a hard mask for an underlying process.

In the case of forming a resist layer from the above photoresist material, a wet process such as spin coating or screen printing is preferably used, as in the case of forming the above underlayer film. After coating with the resist material by spin coating or the like, prebaking is generally performed. This prebaking is preferably performed at 80 to 180° C. in the range of 10 to 300 seconds. Then, exposure, post-exposure baking (PEB), and development can be performed according to a conventional method to obtain a resist pattern. The thickness of the resist film is not particularly limited, and in general, is preferably 30 to 500 nm and more preferably 50 to 400 nm.

The exposure light can be arbitrarily selected and used according to the photoresist material to be used. General examples thereof can include a high energy ray having a wavelength of 300 nm or less, specifically, excimer laser of 248 nm, 193 nm, or 157 nm, soft x-ray of 3 to 20 nm, electron beam, and X-ray.

In a resist pattern formed by the method mentioned above, pattern collapse is suppressed by the underlayer film of the present embodiment. Therefore, use of the underlayer film of the present embodiment can produce a finer pattern and can reduce an exposure amount necessary for obtaining the resist pattern.

Next, etching is performed with the obtained resist pattern as a mask. Gas etching is preferably used as the etching of the underlayer film in a two-layer process. The gas etching is suitably etching using oxygen gas. In addition to oxygen gas, an inert gas such as He or Ar, or CO, CO₂, NH₃, SO₂, N₂, NO₂, or H₂ gas may be added. Alternatively, the gas etching may be performed with CO, CO₂, NH₃, N₂, NO₂, or H₂ gas without the use of oxygen gas. Particularly, the latter gas is preferably used for side wall protection in order to prevent the undercut of pattern side walls.

On the other hand, gas etching is also preferably used as the etching of the intermediate layer in a three-layer process. The same gas etching as described in the two-layer process mentioned above is applicable. Particularly, it is preferable to process the intermediate layer in a three-layer process by using chlorofluorocarbon-based gas and using the resist pattern as a mask. Then, as mentioned above, for example, the underlayer film can be processed by oxygen gas etching with the intermediate layer pattern as a mask.

Here, in the case of forming an inorganic hard mask intermediate layer film as the intermediate layer, a silicon oxide film, a silicon nitride film, or a silicon oxynitride film (SiON film) is formed by CVD, ALD, or the like. A method for forming the nitride film is not limited, and for example, a method described in Japanese Patent Application Laid-Open No. 2002-334869 (Patent Literature 6) or WO 2004/066377 (Patent Literature 7) can be used. Although a photoresist film can be formed directly on such an intermediate layer film, an organic antireflection film (BARC) may be formed on the intermediate layer film by spin coating and a photoresist film may be formed thereon.

A polysilsesquioxane-based intermediate layer is preferably used as the intermediate layer. By imparting effects as an antireflection film to the resist intermediate layer film, there is a tendency that reflection can be effectively suppressed. A specific material for the polysilsesquioxane-based intermediate layer is not limited, and, for example, a material described in Japanese Patent Application Laid-Open No. 2007-226170 (Patent Literature 8) or Japanese Patent Application Laid-Open No. 2007-226204 (Patent Literature 9) can be used.

The subsequent etching of the substrate can also be performed by a conventional method. For example, the substrate made of SiO2 or SiN can be etched mainly using chlorofluorocarbon-based gas, and the substrate made of p-Si, Al, or W can be etched mainly using chlorine- or bromine-based gas. In the case of etching the substrate with chlorofluorocarbon-based gas, the silicon-containing resist of the two-layer resist process or the silicon-containing intermediate layer of the three-layer process is stripped at the same time with substrate processing. On the other hand, in the case of etching the substrate with chlorine- or bromine-based gas, the silicon-containing resist layer or the silicon-containing intermediate layer is separately stripped and in general, stripped by dry etching using chlorofluorocarbon-based gas after substrate processing.

A feature of the underlayer film of the present embodiment is that it is excellent in etching resistance of these substrates. The substrate can be arbitrarily selected from publicly known ones and used and is not particularly limited. Examples thereof include Si, a-Si, p-Si, SiO₂, SiN, SiON, W, TiN, and Al. The substrate may be a laminate having a film to be processed (substrate to be processed) on a base material (support). Examples of such a film to be processed include various low-k films such as Si, SiO₂, SiON, SiN, p-Si, α-Si, W, W—Si, Al, Cu, and Al—Si, and stopper films thereof. A material different from that for the base material (support) is generally used. The thickness of the substrate to be processed or the film to be processed is not particularly limited, and normally, it is preferably approximately 50 to 1,000,000 nm and more preferably 75 to 500,000 nm.

### EXAMPLES

Hereinafter, the present invention will be described in further detail with reference to Synthetic Working Examples, Examples, Production Examples, and Comparative Examples, but the present invention is not limited by these examples in any way.

### [Molecular Weight]

The molecular weight of the synthesized compound was measured by LC-MS analysis using Acquity UPLC/MALDI-Synapt HDMS manufactured by Waters Corporation.

### [Evaluation of Heat Resistance]

EXSTAR 6000 TG-DTA apparatus manufactured by SII NanoTechnology Inc. was used. About 5 mg of a sample was placed in an unsealed container made of aluminum, and the temperature was raised to 500° C. at a temperature increase rate of 10° C./min in a nitrogen gas stream (100 ml/min), thereby measuring the amount of thermogravimetric weight loss. From a practical viewpoint, evaluation A or B described below is preferable. When the evaluation is A or B, the sample has high heat resistance and is applicable to high temperature baking.

### <Evaluation Criteria>

A: The amount of thermogravimetric weight loss at 400° C. is less than 10%

B: The amount of thermogravimetric weight loss at 400° C. is 10% to 25%

C: The amount of thermogravimetric weight loss at 400° C. is greater than 25%

### [Evaluation of Solubility]

Propylene glycol monomethyl ether acetate (PGMEA) and the compound and/or the resin were added to a 50 ml screw bottle and stirred at 23° C. for 1 hour using a magnetic stirrer. Then, the amount of the compound and/or the resin dissolved in PGMEA was measured and the result was evaluated according to the following criteria. From a practical viewpoint, evaluation S, A, or B described below is preferable. When the evaluation is S, A, or B, the sample has high storage stability in the solution state, and can be satisfyingly applied to an edge bead rinse solution (mixed liquid of PGME/PGMEA) widely used for a fine processing process of semiconductors.

### <Evaluation Criteria>

S: 15% by mass or more and less than 35% by mass

A: 5% by mass or more and less than 15% by mass

B: less than 5% by mass

### (Synthetic Working Example 1) Synthesis of BAPP Citramaleimide

A container (internal capacity: 100 ml) equipped with a stirrer, a condenser tube, and a burette was prepared. To this container, 4.10 g (10.0 mmol) of 2,2-bis[4-(4-aminophenoxy)phenyl]propane (product name: BAPP, manufactured by Wakayama Seika Kogyo Co., Ltd.), 2.07 g (20.0 mmol) of citraconic anhydride (manufactured by KANTO CHEMICAL CO., INC.), 2.07 g (20.0 mmol) of maleic anhydride (manufactured by KANTO CHEMICAL CO., INC.), 30 ml of dimethylformamide, and 60 ml of toluene were charged, and 0.4 g (2.3 mmol) of p-toluenesulfonic acid and 0.1 g of a polymerization inhibitor BHT were added, thereby preparing a reaction solution. The reaction solution was stirred at 120° C. for 5 hours to conduct reaction, and the produced water was recovered with a Dean-and-stark trap through azeotropic dehydration. Next, after cooling the reaction solution to 40° C., it was added dropwise into a beaker in which 300 ml of distilled water was placed to precipitate the product. After filtering the obtained slurry solution, the residue was washed with acetone and subjected to separation and purification with column chromatography to acquire 3.8 g of the target compound, citramaleimide, represented by the following formula:

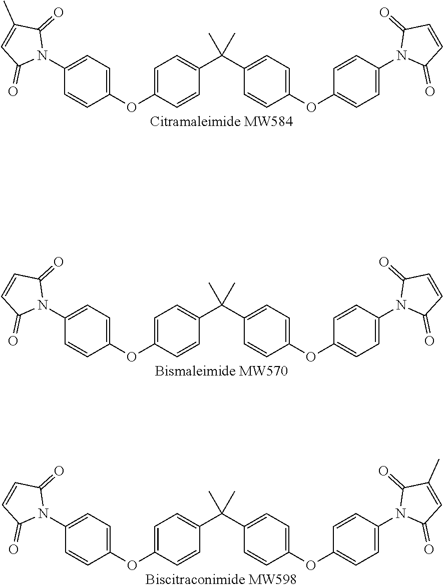 [SMILES: CC1=CC(=O)N(c2ccc(Oc3ccc(C(C)(C)c4ccc(Oc5ccc(N6C(=O)C=CC6=O)cc5)cc4)cc3)cc2)C1=O ; CC(C)(c1ccc(Oc2ccc(N3C(=O)C=CC3=O)cc2)cc1)c1ccc(Oc2ccc(N3C(=O)C=CC3=O)cc2)cc1 ; CC1=CC(=O)N(c2ccc(Oc3ccc(C(C)(C)c4ccc(Oc5ccc(N6C(=O)C=CC6=O)cc5)cc4)cc3)cc2)C1=O]

The following peaks were found by 400 MHz-¹H-NMR, and the citramaleimide was confirmed to have a chemical structure of the above formula.

¹H-NMR: (d-DMSO, internal standard TMS) 6 (ppm) 7.0-7.3 (18.0H, Ph-H, ═CH—), 6.8 (1.0H, ═CH—), 2.0 (3.0H, —CH₃ (citraconimide ring)), 1.7 (6H, —CH₃).

As a result of measuring the molecular weight of the obtained product by the above method, it was found that the product is a mixture of three compounds: 584 (citramaleimide), 570 (bismaleimide), and 598 (biscitraconimide). Also, the composition ratio (584 (citramaleimide)/570 (bismaleimide)/598 (biscitraconimide)) was 50/25/25.

Note that, in the following Examples, a single compound of citramaleimide was used to prepare a film forming material for lithography.

In the following Synthetic Working Examples 2 to 4 as well, the product was obtained as a mixture of citramaleimide/bismaleimide/biscitraconimide in a ratio of 50/25/25, but in Examples, a single compound of citramaleimide was used to prepare a film forming material for lithography.

### Synthetic Working Example 2

### Synthesis of APB-N Citramaleimide

A container (internal capacity: 100 ml) equipped with a stirrer, a condenser tube, and a burette was prepared. To this container, 2.92 g (10.0 mmol) of 3,3′-(1,3-phenylenebis)oxydianiline (product name: APB-N, manufactured by MITSUI FINE CHEMICALS, Inc.), 2.07 g (20.0 mmol) of citraconic anhydride (manufactured by KANTO CHEMICAL CO., INC.), 2.07 g (20.0 mmol) of maleic anhydride (manufactured by KANTO CHEMICAL CO., INC.), 30 ml of dimethylformamide, and 60 ml of toluene were charged, and 0.4 g (2.3 mmol) of p-toluenesulfonic acid and 0.1 g of a polymerization inhibitor BHT were added, thereby preparing a reaction solution. The reaction solution was stirred at 110° C. for 5 hours to conduct reaction and the produced water was recovered with a Dean-and-stark trap through azeotropic dehydration. Next, after cooling the reaction solution to 40° C., it was added dropwise into a beaker in which 300 ml of distilled water was placed to precipitate the product. After filtering the obtained slurry solution, the residue was washed with methanol and subjected to separation and purification with column chromatography to acquire 3.52 g of the target compound (APB-N citramaleimide) represented by the following formula:

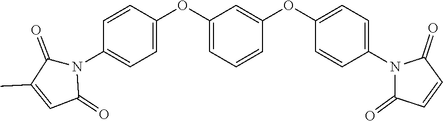 [SMILES: CC1=CC(=O)N(c2ccc(Oc3cccc(Oc4ccc(N5C(=O)C=CC5=O)cc4)c3)cc2)C1=O]

The following peaks were found by 400 MHz-¹H-NMR, and the compound was confirmed to have a chemical structure of the above formula.

¹H-NMR: (d-DMSO, internal standard TMS) δ (ppm) 6.8-7.3 (12H, Ph-H), 7.0 (3H, —CH═C), 2.1 (3H, C—CH₃). As a result of measuring the molecular weight of the obtained compound by the above method, it was 466.

### Synthetic Working Example 3

### Synthesis of HFBAPP Citramaleimide

A container (internal capacity: 100 ml) equipped with a stirrer, a condenser tube, and a burette was prepared. To this container, 5.18 g (10.0 mmol) of 2,2-bis[4-(4-aminophenoxy)phenyl]hexafluoropropane (product name: HFBAPP, manufactured by Wakayama Seika Kogyo Co., Ltd.), 2.27 g (22.0 mmol) of citraconic anhydride (manufactured by KANTO CHEMICAL CO., INC.), 2.27 g (22.0 mmol) of maleic anhydride (manufactured by KANTO CHEMICAL CO., INC.), 30 ml of dimethylformamide, and 60 ml of toluene were charged, and 0.4 g (2.3 mmol) of p-toluenesulfonic acid and 0.1 g of a polymerization inhibitor BHT were added, thereby preparing a reaction solution. The reaction solution was stirred at 110° C. for 5.0 hours to conduct reaction, and the produced water was recovered with a Dean-and-stark trap through azeotropic dehydration. Next, after cooling the reaction solution to 40° C., it was added dropwise into a beaker in which 300 ml of distilled water was placed to precipitate the product. After filtering the obtained slurry solution, the residue was washed with methanol and subjected to separation and purification with column chromatography to acquire 3.9 g of the target compound (HFBAPP citramaleimide) represented by the following formula:

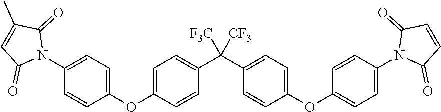 [SMILES: CC1=CC(=O)N(c2ccc(Oc3ccc(C(c4ccc(Oc5ccc(N6C(=O)C=CC6=O)cc5)cc4)(C(F)(F)F)C(F)(F)F)cc3)cc2)C1=O]

The following peaks were found by 400 MHz-¹H-NMR and the compound was confirmed to have a chemical structure of the above formula.

¹H-NMR: (d-DMSO, internal standard TMS) δ (ppm) 6.6-7.35 (16H, Ph-H), 2.1 (3H, C—CH₃), 6.4 (3H, —CH═CH—).

As a result of measuring the molecular weight of the obtained compound by the above method, it was 691.

### Synthetic Working Example 4

### Synthesis of BisAP Citramaleimide

A container (internal capacity: 100 ml) equipped with a stirrer, a condenser tube, and a burette was prepared. To this container, 5.18 g (10.0 mmol) of 1,4-bis[2-(4-aminophenyl)-2-propyl]benzene (product name: Bisaniline-P, manufactured by MITSUI FINE CHEMICALS, Inc.), 2.27 g (22.0 mmol) of citraconic anhydride (manufactured by KANTO CHEMICAL CO., INC.), 2.27 g (22.0 mmol) of maleic anhydride (manufactured by KANTO CHEMICAL CO., INC.), 30 ml of dimethylformamide, and 60 ml of toluene were charged, and 0.4 g (2.3 mmol) of p-toluenesulfonic acid and 0.1 g of a polymerization inhibitor BHT were added, thereby preparing a reaction solution. The reaction solution was stirred at 110° C. for 6.0 hours to conduct reaction, and the produced water was recovered with a Dean-and-stark trap through azeotropic dehydration. Next, after cooling the reaction solution to 40° C., it was added dropwise into a beaker in which 300 ml of distilled water was placed to precipitate the product. After filtering the obtained slurry solution, the residue was washed with methanol and subjected to separation and purification with column chromatography to acquire 4.2 g of the target compound (BisAP citramaleimide) represented by the following formula:

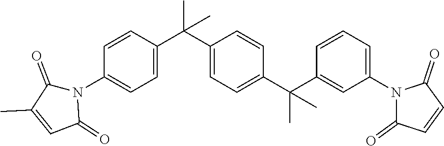 [SMILES: CC1=CC(=O)N(c2ccc(C(C)(C)c3ccc(C(C)(C)c4cccc(N5C(=O)C=CC5=O)c4)cc3)cc2)C1=O]

The following peaks were found by 400 MHz-¹H-NMR, and the compound was confirmed to have a chemical structure of the formula described above.

¹H-NMR: (d-DMSO, internal standard TMS) δ (ppm) 6.8-7.35 (12H, Ph-H), 6.7 (3H, —CH═C), 2.1 (3H, C—CH₃), 1.6-1.7 (12H, —C (CH₃)₂).

As a result of measuring the molecular weight of the obtained compound by the above method, it was 517.

### Synthetic Working Example 5

### Synthesis of BMI Citramaleimide Resin

A container (internal capacity: 100 ml) equipped with a stirrer, a condenser tube, and a burette was prepared. To this container, 2.4 g of diaminodiphenylmethane oligomers obtained by following up on Synthetic Example 1 in Japanese Patent Application Laid-Open No. 2001-26571, a mixture of citraconic anhydride and maleic anhydride (22.0 mmol/22.0 mmol), 40 ml of dimethylformamide, and 60 ml of toluene were charged, and 0.4 g (2.3 mmol) of p-toluenesulfonic acid and 0.1 g of a polymerization inhibitor BHT were added, thereby preparing a reaction solution. The reaction solution was stirred at 110° C. for 8.0 hours to conduct reaction, and the produced water was recovered with a Dean-and-stark trap through azeotropic dehydration. Next, after cooling the reaction solution to 40° C., it was added dropwise into a beaker in which 300 ml of distilled water was placed to precipitate the product. After filtering the obtained slurry solution, the residue was washed with methanol to acquire 4.6 g of a BMI citramaleimide resin.

### Synthetic Working Example 6

### Synthesis of BAN Citramaleimide Resin

A container (internal capacity: 100 ml) equipped with a stirrer, a condenser tube, and a burette was prepared. To this container, 6.30 g of a biphenyl aralkyl-based polyaniline resin (product name: BAN, manufactured by Nippon Kayaku Co., Ltd.), a mixture of citraconic anhydride and maleic anhydride (22.0 mmol/22.0 mmol), 40 ml of dimethylformamide, and 60 ml of toluene were charged, and 0.4 g (2.3 mmol) of p-toluenesulfonic acid and 0.1 g of a polymerization inhibitor BHT were added, thereby preparing a reaction solution. The reaction solution was stirred at 110° C. for 6.0 hours to perform reaction, and the produced water was recovered with a Dean-and-stark trap through azeotropic dehydration. Next, after cooling the reaction solution to 40° C., it was added dropwise into a beaker in which 300 ml of distilled water was placed to precipitate the product. After filtering the obtained slurry solution, the residue was washed with methanol and subjected to separation and purification with column chromatography to acquire 4.6 g of a BAN citramaleimide resin.

### Synthetic Working Example 7

### Synthesis of High Molecular Weight BMI Citramaleimide Polymer

To a 300 mL flask container, 30 g of diaminodiphenylmethane oligomers (DDMO) obtained by following up on Synthetic Example 1 in Japanese Patent Application Laid-Open No. 2001-26571 was charged, and 60 g of methyl ethyl ketone was added as a solvent. By heating the resultant mixture to 60° C. for dissolution, a solution was obtained. The above solution was adsorbed on a neutral silica gel (manufactured by Kanto Chemical Co., Inc.), and by using silica gel column chromatography and developing a mixed solvent of ethyl acetate (20% by mass)/hexane (80% by mass), only the component of the repeating unit represented by the following formula was separated. After concentration, vacuum drying was performed to remove the solvent, thereby obtaining 9.6 g of a high molecular weight DDMO polymer.

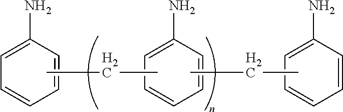

(High molecular weight DDMO polymer; in the formula, n represents an integer of 1 to 4)

To a container (internal capacity: 100 ml) equipped with a stirrer, a condenser tube, and a burette, 4.0 g of the above high molecular weight diaminodiphenylmethane oligomer polymer, a mixture of citraconic anhydride and maleic anhydride (22.0 mmol/22.0 mmol), 40 ml of dimethylformamide, and 60 ml of toluene were charged, and 0.4 g (2.3 mmol) of p-toluenesulfonic acid and 0.1 g of a polymerization inhibitor BHT were added, thereby preparing a reaction solution. The reaction solution was stirred at 110° C. for 8.0 hours to perform reaction, and the produced water was recovered with a Dean-and-stark trap through azeotropic dehydration. Next, after cooling the reaction solution to 40° C., it was added dropwise into a beaker in which 300 ml of distilled water was placed to precipitate the product. After filtering the obtained slurry solution, the residue was washed with methanol to acquire 5.5 g of a high molecular weight BMI citramaleimide polymer.

### Synthetic Working Example 8

### Synthesis of High Molecular Weight BAN Citramaleimide Polymer

To a 300 mL flask container, 40 g of a biphenyl aralkyl-based polyaniline resin (product name: BAN, manufactured by Nippon Kayaku Co., Ltd.) was charged, and 60 g of methyl ethyl ketone was added as a solvent. By heating the resultant mixture to 60° C. for dissolution, a solution was obtained. The above solution was adsorbed on a neutral silica gel (manufactured by Kanto Chemical Co., Inc.), and by using silica gel column chromatography and developing a mixed solvent of ethyl acetate (20% by mass)/hexane (80% by mass), only the component of the repeating unit represented by the following formula was separated. After concentration, vacuum drying was performed to remove the solvent, thereby obtaining 11.6 g of a high molecular weight BAN polymer.

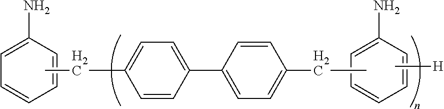

(High molecular weight BAN polymer; in the formula, n represents an integer of 2 to 4)

To a container (internal capacity: 100 ml) equipped with a stirrer, a condenser tube, and a burette, 5.0 g of the above high molecular weight BAN polymer, a mixture of citraconic anhydride and maleic anhydride (22.0 mmol/22.0 mmol), 40 ml of dimethylformamide, and 60 ml of toluene were charged, and 0.4 g (2.3 mmol) of p-toluenesulfonic acid and 0.1 g of a polymerization inhibitor BHT were added, thereby preparing a reaction solution. The reaction solution was stirred at 110° C. for 8.0 hours to perform reaction and the produced water was recovered with a Dean-and-stark trap through azeotropic dehydration. Next, after cooling the reaction solution to 40° C., it was added dropwise into a beaker in which 300 ml of distilled water was placed to precipitate the product. After filtering the obtained slurry solution, the residue was washed with methanol to acquire 6.6 g of a high molecular weight BAN citramaleimide polymer.

### Example 1

The BAPP citramaleimide obtained in Synthetic Working Example 1 was used as a film forming material for lithography.

As a result of thermogravimetry, the amount of thermogravimetric weight loss at 400° C. of the obtained film forming material for lithography was less than 10% (evaluation A). In addition, as a result of evaluation of solubility in PGMEA, the solubility was 5% by mass or more and less than 15% by mass (evaluation A), and the obtained film forming material for lithography was evaluated to have sufficient solubility.

By using 5 parts by mass of the BAPP citramaleimide obtained in Synthetic Working Example 1, that is, 5 parts by mass of the above film forming material for lithography, adding 95 parts by mass of propylene glycol monomethyl ether acetate (PGMEA) as a solvent thereto, and stirring the resultant mixture with a stirrer at room temperature for at least 3 hours, a composition for film formation for lithography was prepared.

### Example 2

The APB-N citramaleimide obtained in Synthetic Working Example 2 was used as a film forming material for lithography.

As a result of thermogravimetry, the amount of thermogravimetric weight loss at 400° C. of the obtained film forming material for lithography was less than 10% (evaluation A). In addition, as a result of evaluation of solubility in PGMEA, the solubility was 5% by mass or more and less than 15% by mass (evaluation A) and the obtained film forming material for lithography was evaluated to have sufficient solubility.

The same operations as in the above Example 1 were carried out, thereby preparing a composition for film formation for lithography.

### Example 3

The HFBAPP citramaleimide obtained in Synthetic Working Example 3 was used as a film forming material for lithography.

As a result of thermogravimetry, the amount of thermogravimetric weight loss at 400° C. of the obtained film forming material for lithography was less than 10% (evaluation A). Also, as a result of evaluation of solubility in PGMEA, the solubility was 5% by mass or more and less than 15% by mass (evaluation A), and the obtained film forming material for lithography was evaluated to have sufficient solubility.

The same operations as in the above Example 1 were carried out, thereby preparing a composition for film formation for lithography.

### Example 4

The BisAP citramaleimide obtained in Synthetic Working Example 4 was used as a film forming material for lithography.

As a result of thermogravimetry, the amount of thermogravimetric weight loss at 400° C. of the obtained film forming material for lithography was less than 10% (evaluation A). Also, as a result of evaluation of solubility in PGMEA, the solubility was 5% by mass or more and less than 15% by mass (evaluation A) and the obtained film forming material for lithography was evaluated to have sufficient solubility.

The same operations as in the above Example 1 were carried out, thereby preparing a composition for film formation for lithography.

### Example 5

The BMI citramaleimide resin obtained in Synthetic Working Example 5 was used as a film forming material for lithography.

As a result of thermogravimetry, the amount of thermogravimetric weight loss at 400° C. of the obtained film forming material for lithography was less than 10% (evaluation A). Moreover, as a result of evaluation of solubility in PGMEA, the solubility was 5% by mass or more and less than 15% by mass (evaluation A), and the obtained film forming material for lithography was evaluated to have sufficient solubility.

The same operations as in the above Example 1 were carried out, thereby preparing a composition for film formation for lithography.

### Example 5A

The high molecular weight BMI citramaleimide polymer obtained in Synthetic Working Example 7 was used as a film forming material for lithography.

As a result of thermogravimetry, the amount of thermogravimetric weight loss at 400° C. of the obtained film forming material for lithography was less than 10% (evaluation A). Moreover, as a result of evaluation of solubility in PGMEA, the solubility was 5% by mass or more and less than 15% by mass (evaluation A) and the obtained film forming material for lithography was evaluated to have sufficient solubility.

The same operations as in the above Example 1 were carried out, thereby preparing a composition for film formation for lithography.

### Example 6

The BAN citramaleimide resin obtained in Synthetic Working Example 6 was used as a film forming material for lithography.

As a result of thermogravimetry, the amount of thermogravimetric weight loss at 400° C. of the obtained film forming material for lithography was less than 10% (evaluation A). Furthermore, as a result of evaluation of solubility in PGMEA, the solubility was 5% by mass or more and less than 15% by mass (evaluation A), and the obtained film forming material for lithography was evaluated to have sufficient solubility.

The same operations as in the above Example 1 were carried out, thereby preparing a composition for film formation for lithography.

### Example 6A

The high molecular weight BAN citramaleimide polymer obtained in Synthetic Working Example 8 was used as a film forming material for lithography.

As a result of thermogravimetry, the amount of thermogravimetric weight loss at 400° C. of the obtained film forming material for lithography was less than 10% (evaluation A). Furthermore, as a result of evaluation of solubility in PGMEA, the solubility was 5% by mass or more and less than 15% by mass (evaluation A) and the obtained film forming material for lithography was evaluated to have sufficient solubility.

The same operations as in the above Example 1 were carried out, thereby preparing a composition for film formation for lithography.

### Example 7

5 parts by mass of the BAPP citramaleimide and 0.1 parts by mass of TPIZ as a crosslinking promoting agent were compounded to prepare a film forming material for lithography.

As a result of thermogravimetry, the amount of thermogravimetric weight loss at 400° C. of the obtained film forming material for lithography was less than 10% (evaluation A). In addition, as a result of evaluation of solubility in PGMEA, the solubility was 5% by mass or more and less than 15% by mass (evaluation A), and it was evaluated that the obtained film forming material for lithography has sufficient solubility.

A composition for film formation for lithography was prepared by the same operations as in the above Example 1 except that the above film forming material for lithography was used.

### Example 8

5 parts by mass of the APB-N citramaleimide and 0.1 parts by mass of TPIZ as a crosslinking promoting agent were compounded to prepare a film forming material for lithography.

As a result of thermogravimetry, the amount of thermogravimetric weight loss at 400° C. of the obtained film forming material for lithography was less than 10% (evaluation A). In addition, as a result of evaluation of solubility in PGMEA, the solubility was 5% by mass or more and less than 15% by mass (evaluation A) and it was evaluated that the obtained film forming material for lithography has sufficient solubility.

A composition for film formation for lithography was prepared by the same operations as in the above Example 1, except that the above film forming material for lithography was used.

### Example 9

5 parts by mass of the HFBAPP citramaleimide and 0.1 parts by mass of TPIZ as a crosslinking promoting agent were compounded to prepare a film forming material for lithography.

As a result of thermogravimetry, the amount of thermogravimetric weight loss at 400° C. of the obtained film forming material for lithography was less than 10% (evaluation A). Also, as a result of evaluation of solubility in PGMEA, the solubility was 5% by mass or more and less than 15% by mass (evaluation A), and it was evaluated that the obtained film forming material for lithography has sufficient solubility.

Except that the above film forming material for lithography was used, a composition for film formation for lithography was prepared by the same operations as in the above Example 1.

### Example 10>

5 parts by mass of the BisAP citramaleimide and 0.1 parts by mass of TPIZ as a crosslinking promoting agent were compounded to prepare a film forming material for lithography.

As a result of thermogravimetry, the amount of thermogravimetric weight loss at 400° C. of the obtained film forming material for lithography was less than 10% (evaluation A). Also, as a result of evaluation of solubility in PGMEA, the solubility was 5% by mass or more and less than 15% by mass (evaluation A) and it was evaluated that the obtained film forming material for lithography has sufficient solubility.

Except that the above film forming material for lithography was used, a composition for film formation for lithography was prepared according to the same operations as in the above Example 1.

### Example 11>

5 parts by mass of the BMI citramaleimide resin and 0.1 parts by mass of TPIZ as a crosslinking promoting agent were compounded to prepare a film forming material for lithography.

As a result of thermogravimetry, the amount of thermogravimetric weight loss at 400° C. of the obtained film forming material for lithography was less than 10% (evaluation A). Moreover, as a result of evaluation of solubility in PGMEA, the solubility was 5% by mass or more and less than 15% by mass (evaluation A), and it was evaluated that the obtained film forming material for lithography has sufficient solubility.

A composition for film formation for lithography was prepared according to the same operations as in the above Example 1, except that the above film forming material for lithography was used.

### Example 11A

5 parts by mass of the high molecular weight BMI citramaleimide polymer and 0.1 parts by mass of TPIZ as a crosslinking promoting agent were compounded to prepare a film forming material for lithography.

As a result of thermogravimetry, the amount of thermogravimetric weight loss at 400° C. of the obtained film forming material for lithography was less than 10% (evaluation A). Moreover, as a result of evaluation of solubility in PGMEA, the solubility was 5% by mass or more and less than 15% by mass (evaluation A) and it was evaluated that the obtained film forming material for lithography has sufficient solubility.

A composition for film formation for lithography was prepared according to the same operations as in the above Example 1 except that the above film forming material for lithography was used.

### Example 12

5 parts by mass of the BAN citramaleimide resin and 0.1 parts by mass of TPIZ as a crosslinking promoting agent were compounded to prepare a film forming material for lithography.

As a result of thermogravimetry, the amount of thermogravimetric weight loss at 400° C. of the obtained film forming material for lithography was less than 10% (evaluation A). Furthermore, as a result of evaluation of solubility in PGMEA, the solubility was 5% by mass or more and less than 15% by mass (evaluation A), and it was evaluated that the obtained film forming material for lithography has sufficient solubility.

A composition for film formation for lithography was prepared according to the same operations as in the Example 1 described above except that the above film forming material for lithography was used.

### Example 12A

5 parts by mass of the high molecular weight BAN citramaleimide polymer and 0.1 parts by mass of TPIZ as a crosslinking promoting agent were compounded to prepare a film forming material for lithography.

As a result of thermogravimetry, the amount of thermogravimetric weight loss at 400° C. of the obtained film forming material for lithography was less than 10% (evaluation A). Furthermore, as a result of evaluation of solubility in PGMEA, the solubility was 5% by mass or more and less than 15% by mass (evaluation A) and it was evaluated that the obtained film forming material for lithography has sufficient solubility.

A composition for film formation for lithography was prepared according to the same operations as in the Example 1 described above, except that the above film forming material for lithography was used.

### Example 13

In addition to 5 parts by mass of the BAPP citramaleimide, 2 parts by mass of benzoxazine (BF-BXZ; manufactured by KONISHI CHEMICAL IND. CO., LTD.) represented by the formula described below was also used as the crosslinking agent, and 0.1 parts by mass of 2,4,5-triphenylimidazole (TPIZ) was compounded as the crosslinking promoting agent to prepare a film forming material for lithography.

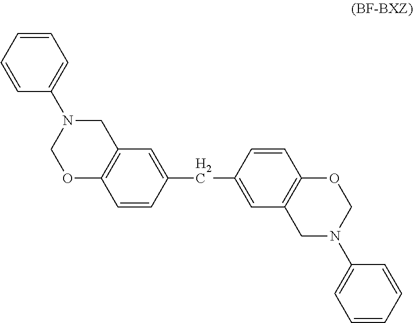 [SMILES: c1ccc(N2COc3ccc(Cc4ccc5c(c4)CN(c4ccccc4)CO5)cc3C2)cc1]

As a result of thermogravimetry, the amount of thermogravimetric weight loss at 400° C. of the obtained film forming material for lithography was less than 10% (evaluation A). In addition, as a result of evaluation of solubility in PGMEA, the solubility was 5% by mass or more and less than 15% by mass (evaluation A), and it was evaluated that the obtained film forming material for lithography has sufficient solubility.

A composition for film formation for lithography was prepared by the same operations as in the Example 1 described above, except that the above film forming material for lithography was used.

### Example 14

In addition to 5 parts by mass of the BAPP citramaleimide, 2 parts by mass of a biphenyl aralkyl-based epoxy resin (NC-3000-L; manufactured by Nippon Kayaku Co., Ltd.) represented by the formula described below was also used as the crosslinking agent, and 0.1 parts by mass of TPIZ was compounded as the crosslinking promoting agent to prepare a film forming material for lithography.

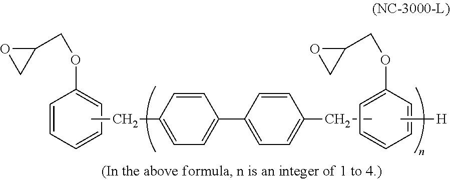

As a result of thermogravimetry, the amount of thermogravimetric weight loss at 400° C. of the obtained film forming material for lithography was less than 10% (evaluation A). In addition, as a result of evaluation of solubility in PGMEA, the solubility was 5% by mass or more and less than 15% by mass (evaluation A) and it was evaluated that the obtained film forming material for lithography has sufficient solubility.

A composition for film formation for lithography was prepared by the same operations as in the Example 1 described above except that the above film forming material for lithography was used.

### Example 15

In addition to 5 parts by mass of the BAPP citramaleimide, 2 parts by mass of a diallylbisphenol A-based cyanate (DABPA-CN; manufactured by MITSUBISHI GAS CHEMICAL COMPANY, INC.) represented by the formula described below was also used as the crosslinking agent, and 0.1 parts by mass of 2,4,5-triphenylimidazole (TPIZ) was compounded as the crosslinking promoting agent to prepare a film forming material for lithography.

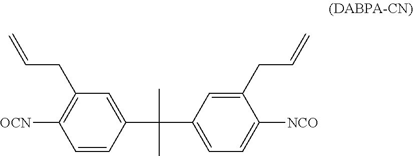

As a result of thermogravimetry, the amount of thermogravimetric weight loss at 400° C. of the obtained film forming material for lithography was less than 10% (evaluation A). Also, as a result of evaluation of solubility in PGMEA, the solubility was 5% by mass or more and less than 15% by mass (evaluation A), and it was evaluated that the obtained film forming material for lithography has sufficient solubility.

Except that the above film forming material for lithography was used, a composition for film formation for lithography was prepared by the same operations as in the Example 1 described above.

### Example 16

In addition to 5 parts by mass of the BAPP citramaleimide, 2 parts by mass of diallylbisphenol A (BPA-CA; manufactured by KONISHI CHEMICAL IND. CO., LTD.) represented by the formula described below was also used as the crosslinking agent, and 0.1 parts by mass of 2,4,5-triphenylimidazole (TPIZ) was compounded as the crosslinking promoting agent to prepare a film forming material for lithography.

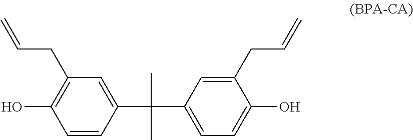 [SMILES: C=CCc1cc(C(C)(C)c2ccc(O)c(CC=C)c2)ccc1O]

As a result of thermogravimetry, the amount of thermogravimetric weight loss at 400° C. of the obtained film forming material for lithography was less than 10% (evaluation A). Also, as a result of evaluation of solubility in PGMEA, the solubility was 5% by mass or more and less than 15% by mass (evaluation A) and it was evaluated that the obtained film forming material for lithography has sufficient solubility.

Except that the above film forming material for lithography was used, a composition for film formation for lithography was prepared through the same operations as in the Example 1 described above.

### Example 17

In addition to 5 parts by mass of the BAPP citramaleimide, 2 parts by mass of a diphenylmethane-based allylphenolic resin (APG-1; manufactured by Gun Ei Chemical Industry Co., Ltd.) represented by the formula described below was also used as the crosslinking agent to prepare a film forming material for lithography.

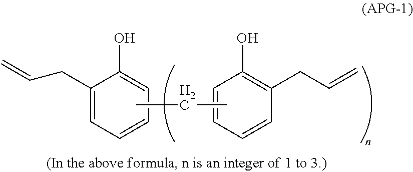

As a result of thermogravimetry, the amount of thermogravimetric weight loss at 400° C. of the obtained film forming material for lithography was less than 10% (evaluation A). Moreover, as a result of evaluation of solubility in PGMEA, the solubility was 5% by mass or more and less than 15% by mass (evaluation A), and it was evaluated that the obtained film forming material for lithography has sufficient solubility.

A composition for film formation for lithography was prepared through the same operations as in the Example 1 described above, except that the above film forming material for lithography was used.

### Example 18

In addition to 5 parts by mass of the BAPP citramaleimide, 2 parts by mass of a diphenylmethane-based propenylphenolic resin (APG-2; manufactured by Gun Ei Chemical Industry Co., Ltd.) represented by the formula described below was also used as the crosslinking agent to prepare a film forming material for lithography.

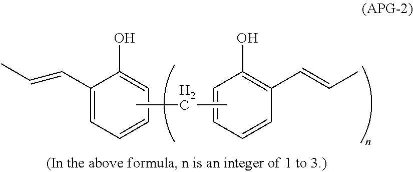

As a result of thermogravimetry, the amount of thermogravimetric weight loss at 400° C. of the obtained film forming material for lithography was less than 10% (evaluation A). Moreover, as a result of evaluation of solubility in PGMEA, the solubility was 5% by mass or more and less than 15% by mass (evaluation A) and it was evaluated that the obtained film forming material for lithography has sufficient solubility.

A composition for film formation for lithography was prepared through the same operations as in the Example 1 described above except that the above film forming material for lithography was used.

### Example 19

In addition to 5 parts by mass of the BAPP citramaleimide, 2 parts by mass of 4,4′-diaminodiphenylmethane (DDM; manufactured by Tokyo Chemical Industry Co., Ltd.) represented by the formula described below was also used as the crosslinking agent to prepare a film forming material for lithography.

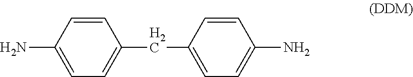 [SMILES: Nc1ccc(Cc2ccc(N)cc2)cc1]

As a result of thermogravimetry, the amount of thermogravimetric weight loss at 400° C. of the obtained film forming material for lithography was less than 10% (evaluation A). Furthermore, as a result of evaluation of solubility in PGMEA, the solubility was 5% by mass or more and less than 15% by mass (evaluation A), and it was evaluated that the obtained film forming material for lithography has sufficient solubility.

A composition for film formation for lithography was prepared through the same operations as in the above Example 1, except that the above film forming material for lithography was used.

### Production Example 1

A four necked flask (internal capacity: 10 L) equipped with a Dimroth condenser tube, a thermometer, and a stirring blade, and having a detachable bottom was prepared. To this four necked flask, 1.09 kg (7 mol) of 1,5-dimethylnaphthalene (manufactured by Mitsubishi Gas Chemical Company, Inc.), 2.1 kg (28 mol as formaldehyde) of a 40 mass % aqueous formalin solution (manufactured by Mitsubishi Gas Chemical Company, Inc.), and 0.97 ml of a 98 mass % sulfuric acid (manufactured by Kanto Chemical Co., Inc.) were added in a nitrogen stream, and the mixture was allowed to react for 7 hours while being refluxed at 100° C. at normal pressure. Subsequently, 1.8 kg of ethylbenzene (manufactured by Wako Pure Chemical Industries, Ltd., a special grade reagent) was added as a diluting solvent to the reaction solution, and the mixture was left to stand still, followed by removal of an aqueous phase as a lower phase. Neutralization and washing with water were further performed, and ethylbenzene and unreacted 1,5-dimethylnaphthalene were distilled off under reduced pressure to obtain 1.25 kg of a dimethylnaphthalene formaldehyde resin as a light brown solid.

The molecular weight of the obtained dimethylnaphthalene formaldehyde resin was as follows: number average molecular weight (Mn): 562, weight average molecular weight (Mw): 1168, and dispersity (Mw/Mn): 2.08.

Subsequently, a four necked flask (internal capacity: 0.5 L) equipped with a Dimroth condenser tube, a thermometer, and a stirring blade was prepared. To this four necked flask, 100 g (0.51 mol) of the dimethylnaphthalene formaldehyde resin obtained as mentioned above, and 0.05 g of p-toluenesulfonic acid were added in a nitrogen stream, and the temperature was raised to 190° C. at which the mixture was then heated for 2 hours, followed by stirring. Subsequently, 52.0 g (0.36 mol) of 1-naphthol was further added thereto, and the temperature was further raised to 220° C. at which the mixture was allowed to react for 2 hours. After dilution with a solvent, neutralization and washing with water were performed, and the solvent was distilled off under reduced pressure to obtain 126.1 g of a modified resin (CR-1) as a black-brown solid.

The obtained resin (CR-1) had Mn: 885, Mw: 2220, and Mw/Mn: 2.51.

As a result of thermogravimetry (TG), the amount of thermogravimetric weight loss at 400° C. of the obtained resin was greater than 25% (evaluation C). Therefore, it was evaluated that application to high temperature baking was difficult.

As a result of evaluation of solubility in PGMEA, the solubility was 10% by mass or more (evaluation A), and the obtained resin was evaluated to have sufficient solubility.

Note that the Mn, Mw, and Mw/Mn described above were measured by carrying out gel permeation chromatography (GPC) analysis under the following conditions to determine the molecular weight in terms of polystyrene.

Apparatus: Shodex GPC-101 model (manufactured by SHOWA DENKO K.K.)

Column: KF-80M×3

Eluent: 1 mL/min THF

Temperature: 40° C.

### Production Example 2

### Synthesis of BAPP Citraconimide

A container (internal capacity: 100 ml) equipped with a stirrer, a condenser tube, and a burette was prepared. To this container, 4.10 g (10.0 mmol) of 2,2-bis[4-(4-aminophenoxy)phenyl]propane (product name: BAPP, manufactured by Wakayama Seika Kogyo Co., Ltd.), 4.15 g (40.0 mmol) of citraconic anhydride (manufactured by KANTO CHEMICAL CO., INC.), 30 ml of dimethylformamide, and 60 ml of toluene were charged, and 0.4 g (2.3 mmol) of p-toluenesulfonic acid and 0.1 g of a polymerization inhibitor BHT were added, thereby preparing a reaction solution. The reaction solution was stirred at 120° C. for 5 hours to conduct reaction, and the produced water was recovered with a Dean-and-stark trap through azeotropic dehydration. Next, after cooling the reaction solution to 40° C., it was added dropwise into a beaker in which 300 ml of distilled water was placed to precipitate the product. After filtering the obtained slurry solution, the residue was washed with acetone and subjected to separation and purification with column chromatography to acquire 3.76 g of the target compound (BAPP citraconimide) represented by the following formula:

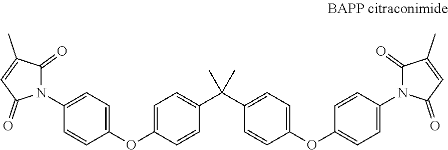 [SMILES: CC1=CC(=O)N(c2ccc(Oc3ccc(C(C)(C)c4ccc(Oc5ccc(N6C(=O)C=C(C)C6=O)cc5)cc4)cc3)cc2)C1=O]

The following peaks were found by 400 MHz-¹H-NMR and the compound was confirmed to have a chemical structure of the formula described above.

¹H-NMR: (d-DMSO, internal standard TMS) δ (ppm) 6.8-7.4 (16H, Ph-H), 6.7 (2H, —CH═C), 2.1 (6H, C—CH₃), 1.6 (6H, —C (CH₃)₂).

As a result of measuring the molecular weight of the obtained compound by the above method, it was 598.

### Production Example 3

### Synthesis of APB-N Citraconimide

A container (internal capacity: 100 ml) equipped with a stirrer, a condenser tube, and a burette was prepared. To this container, 2.92 g (10.0 mmol) of 3,3′-(1, 3-phenylenebis)oxydianiline (product name: APB-N, manufactured by MITSUI FINE CHEMICALS, Inc.), 4.15 g (40.0 mmol) of citraconic anhydride (manufactured by KANTO CHEMICAL CO., INC.), 30 ml of dimethylformamide, and 60 ml of toluene were charged, and 0.4 g (2.3 mmol) of p-toluenesulfonic acid and 0.1 g of a polymerization inhibitor BHT were added, thereby preparing a reaction solution. The reaction solution was stirred at 110° C. for 5 hours to conduct reaction, and the produced water was recovered with a Dean-and-stark trap through azeotropic dehydration. Next, after cooling the reaction solution to 40° C., it was added dropwise into a beaker in which 300 ml of distilled water was placed to precipitate the product. After filtering the obtained slurry solution, the residue was washed with methanol and subjected to separation and purification with column chromatography to acquire 3.52 g of the target compound (APB-N citraconimide) represented by the following formula:

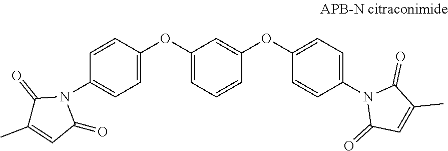

Note that the following peaks were found by 400 MHz-¹H-NMR and the compound was confirmed to have a chemical structure of the formula described above.

¹H-NMR: (d-DMSO, internal standard TMS) δ (ppm) 6.7-7.4 (12H, Ph-H), 6.4 (2H, —CH═C) , 2.2 (6H, C—CH₃. As a result of measuring the molecular weight of the obtained compound by the above method, it was 480.

### Production Example 4

### Synthesis of HFBAPP Citraconimide

A container (internal capacity: 100 ml) equipped with a stirrer, a condenser tube, and a burette was prepared. To this container, 5.18 g (10.0 mmol) of 2,2-bis[4-(4-aminophenoxy)phenyl]hexafluoropropane (product name: HFBAPP, manufactured by Wakayama Seika Kogyo Co., Ltd.), 4.56 g (44.0 mmol) of citraconic anhydride (manufactured by KANTO CHEMICAL CO., INC.), 30 ml of dimethylformamide, and 60 ml of toluene were charged, and 0.4 g (2.3 mmol) of p-toluenesulfonic acid and 0.1 g of a polymerization inhibitor BHT were added, thereby preparing a reaction solution. The reaction solution was stirred at 110° C. for 5.0 hours to conduct reaction and the produced water was recovered with a Dean-and-stark trap through azeotropic dehydration. Next, after cooling the reaction solution to 40° C., it was added dropwise into a beaker in which 300 ml of distilled water was placed to precipitate the product. After filtering the obtained slurry solution, the residue was washed with methanol and subjected to separation and purification with column chromatography to acquire 3.9 g of the target compound (HFBAPP citraconimide) represented by the following formula:

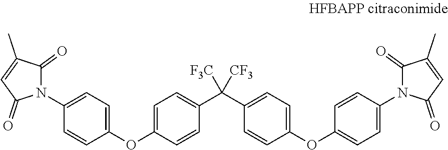 [SMILES: CC1=CC(=O)N(c2ccc(Oc3ccc(C(c4ccc(Oc5ccc(N6C(=O)C=C(C)C6=O)cc5)cc4)(C(F)(F)F)C(F)(F)F)cc3)cc2)C1=O]

Note that the following peaks were found by 400 MHz-¹H-NMR and the compound was confirmed to have a chemical structure of the above formula.

¹H-NMR: (d-DMSO, internal standard TMS) δ (ppm) 6.6-7.3 (16H, Ph-H), 6.4 (2H, —CH═C) , 2.2 (6H, C—CH₃).

As a result of measuring the molecular weight of the obtained compound by the above method, it was 706.

### Production Example 5

### Synthesis of BisAP Citraconimide

A container (internal capacity: 100 ml) equipped with a stirrer, a condenser tube, and a burette was prepared. To this container, 5.18 g (10.0 mmol) of 1,4-bis[2-(4-aminophenyl)-2-propyl]benzene (product name: Bisaniline-P, manufactured by MITSUI FINE CHEMICALS, Inc.), 4.56 g (44.0 mmol) of citraconic anhydride (manufactured by KANTO CHEMICAL CO., INC.), 30 ml of dimethylformamide, and 60 ml of toluene were charged, and 0.4 g (2.3 mmol) of p-toluenesulfonic acid and 0.1 g of a polymerization inhibitor BHT were added, thereby preparing a reaction solution. The reaction solution was stirred at 110° C. for 6.0 hours to perform reaction and the produced water was recovered with a Dean-and-stark trap through azeotropic dehydration. Next, after cooling the reaction solution to 40° C., it was added dropwise into a beaker in which 300 ml of distilled water was placed to precipitate the product. After filtering the obtained slurry solution, the residue was washed with methanol and subjected to separation and purification with column chromatography to acquire 4.2 g of the target compound

(BisAP citraconimide) represented by the following formula:

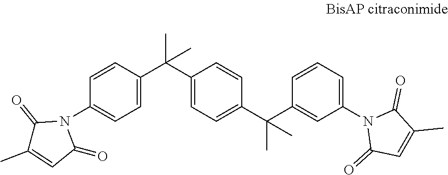

Note that the following peaks were found by 400 MHz-¹H-NMR, and the compound was confirmed to have a chemical structure of the above formula.

¹H-NMR: (d-DMSO, internal standard TMS) δ (ppm) 6.8-7.4 (12H, Ph-H), 6.7 (2H, —CH=C), 2.1 (6H, C-CH₃), 1.6-1.7 (12H, —C (CH₃)₂).

As a result of measuring the molecular weight of the obtained compound by the above method, it was 532.

### Production Example 6

### Synthesis of BMI Citraconimide Resin

A container (internal capacity: 100 ml) equipped with a stirrer, a condenser tube, and a burette was prepared. To this container, 2.4 g of diaminodiphenylmethane oligomers obtained by following up on Synthetic Example 1 in Japanese Patent Application Laid-Open No. 2001-26571, 4.56 g (44.0 mmol) of citraconic anhydride (manufactured by KANTO CHEMICAL CO., INC.), 40 ml of dimethylformamide, and 60 ml of toluene were charged, and 0.4 g (2.3 mmol) of p-toluenesulfonic acid and 0.1 g of a polymerization inhibitor BHT were added, thereby preparing a reaction solution. The reaction solution was stirred at 110° C. for 8.0 hours to conduct reaction and the produced water was recovered with a Dean-and-stark trap through azeotropic dehydration. Next, after cooling the reaction solution to 40° C., it was added dropwise into a beaker in which 300 ml of distilled water was placed to precipitate the product. After filtering the obtained slurry solution, the residue was washed with methanol to acquire 4.7 g of a citraconimide resin (BMI citraconimide resin) represented by the following formula:

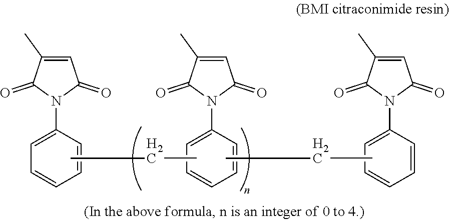

Note that, as a result of measuring the molecular weight by the above method, it was 446.

### Production Example 7

### Synthesis of BAN Citraconimide Resin

A container (internal capacity: 100 ml) equipped with a stirrer, a condenser tube, and a burette was prepared. To this container, 6.30 g of a biphenyl aralkyl-based polyaniline resin (product name: BAN, manufactured by Nippon Kayaku Co., Ltd.), 4.56 g (44.0 mmol) of citraconic anhydride (manufactured by KANTO CHEMICAL CO., INC.), 40 ml of dimethylformamide, and 60 ml of toluene were charged, and 0.4 g (2.3 mmol) of p-toluenesulfonic acid and 0.1 g of a polymerization inhibitor BHT were added, thereby preparing a reaction solution. The reaction solution was stirred at 110° C. for 6.0 hours to carry out reaction, and the produced water was recovered with a Dean-and-stark trap through azeotropic dehydration. Next, after cooling the reaction solution to 40° C., it was added dropwise into a beaker in which 300 ml of distilled water was placed to precipitate the product. After filtering the obtained slurry solution, the residue was washed with methanol and subjected to separation and purification with column chromatography to acquire 5.5 g of the target compound (BAN citraconimide resin) represented by the following formula:

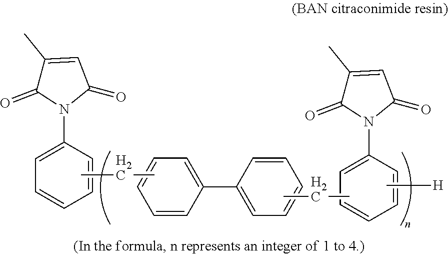

### Comparative Example 1

In addition to 5 parts by mass of CR-1, 2 parts by mass of a biphenyl aralkyl-based epoxy resin (NC-3000-L; manufactured by Nippon Kayaku Co., Ltd.) represented by the formula described below was also used as the crosslinking agent, and 0.1 parts by mass of TPIZ was compounded as the crosslinking promoting agent to prepare a film forming material for lithography.

A composition for film formation for lithography was prepared through the same operations as in the above Example 1 except that the above film forming material for lithography was used.

### Comparative Example 2

CR-1 was used as a film forming material for lithography.

Except that the above film forming material for lithography was used, a composition for film formation for lithography was prepared through the same operations as in the above Example 1.

### Comparative Example 3

The BAPP citraconimide was used as a film forming material for lithography.

As a result of thermogravimetry, the amount of thermogravimetric weight loss at 400° C. of the obtained film forming material for lithography was less than 10% (evaluation A). In addition, as a result of evaluation of solubility in PGMEA, the solubility was 15% by mass or more and less than 35% by mass (evaluation S), and the obtained film forming material for lithography was evaluated to have sufficient solubility.

The same operations as in the above Example 1 were carried out, thereby preparing a composition for film formation for lithography.

### Comparative Example 4

The APB-N citraconimide was used as a film forming material for lithography.

As a result of thermogravimetry, the amount of thermogravimetric weight loss at 400° C. of the obtained film forming material for lithography was less than 10% (evaluation A). In addition, as a result of evaluation of solubility in PGMEA, the solubility was 15% by mass or more and less than 35% by mass (evaluation S) and the obtained film forming material for lithography was evaluated to have sufficient solubility.

The same operations as in the above Example 1 were carried out, thereby preparing a composition for film formation for lithography.

### Comparative Example 5

The HFBAPP citraconimide was used as a film forming material for lithography.

As a result of thermogravimetry, the amount of thermogravimetric weight loss at 400° C. of the obtained film forming material for lithography was less than 10% (evaluation A). Also, as a result of evaluation of solubility in PGMEA, the solubility was 15% by mass or more and less than 35% by mass (evaluation S), and the obtained film forming material for lithography was evaluated to have sufficient solubility.

The same operations as in the above Example 1 were carried out, thereby preparing a composition for film formation for lithography.

### Comparative Example 6

The BisAP citraconimide was used as a film forming material for lithography.

As a result of thermogravimetry, the amount of thermogravimetric weight loss at 400° C. of the obtained film forming material for lithography was less than 10% (evaluation A). Also, as a result of evaluation of solubility in PGMEA, the solubility was 15% by mass or more and less than 35% by mass (evaluation S) and the obtained film forming material for lithography was evaluated to have sufficient solubility.

The same operations as in the above Example 1 were carried out, thereby preparing a composition for film formation for lithography.

### Comparative Example 7

The BMI citraconimide resin was used as a film forming material for lithography.

As a result of thermogravimetry, the amount of thermogravimetric weight loss at 400° C. of the obtained film forming material for lithography was less than 10% (evaluation A). In addition, as a result of evaluation of solubility in PGMEA, the solubility was 5% by mass or more and less than 15% by mass (evaluation A) and it was evaluated that the obtained film forming material for lithography has sufficient solubility.

The same operations as in the above Example 1 were carried out, thereby preparing a composition for film formation for lithography.

### Comparative Example 8

The BAM citraconimide resin was used as a film forming material for lithography.

As a result of thermogravimetry, the amount of thermogravimetric weight loss at 400° C. of the obtained film forming material for lithography was less than 10% (evaluation A). In addition, as a result of evaluation of solubility in PGMEA, the solubility was 5% by mass or more and less than 15% by mass (evaluation A), and it was evaluated that the obtained film forming material for lithography has sufficient solubility.

The same operations as in the above Example 1 were carried out, thereby preparing a composition for film formation for lithography.

### Comparative Example 9

A phenylmethane maleimide oligomer (BMI oligomer; BMI-2300; manufactured by Daiwa Kasei Industry Co., Ltd.) represented by the formula described below was used to prepare a film forming material for lithography.

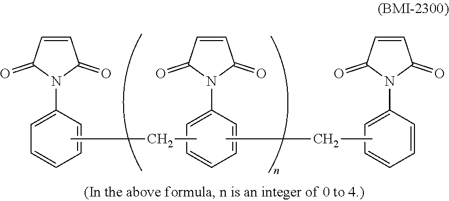

As a result of thermogravimetry, the amount of thermogravimetric weight loss at 400° C. of the obtained film forming material for lithography was less than 10% (evaluation A). In addition, as a result of evaluation of solubility in PGMEA, the solubility was 5% by mass or more and less than 15% by mass (evaluation A), and it was evaluated that the obtained film forming material for lithography has sufficient solubility. The same operations as in the above Example 1 were carried out, thereby preparing a composition for film formation for lithography.

### Comparative Example 10

As a bismaleimide compound, bismaleimide (BMI-80; manufactured by K.I Chemical Industry Co., LTD.) represented by the formula described below was used to prepare a film forming material for lithography.

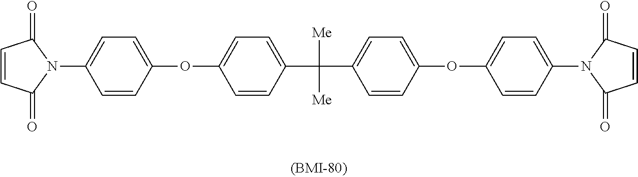 [SMILES: CC(C)(c1ccc(Oc2ccc(N3C(=O)C=CC3=O)cc2)cc1)c1ccc(Oc2ccc(N3C(=O)C=CC3=O)cc2)cc1]

As a result of thermogravimetry, the amount of thermogravimetric weight loss at 400° C. of the obtained film forming material for lithography was less than 10% (evaluation A). Besides, as a result of evaluation of solubility in PGMEA, the solubility was 5% by mass or more and less than 15% by mass (evaluation A), and it was evaluated that the obtained film forming material for lithography has sufficient solubility.

The same operations as in the above Example 1 were carried out, thereby preparing a composition for film formation for lithography.

### Example 20

In addition to 5 parts by mass of the BAPP citramaleimide, 0.1 parts by mass of IRGACURE 184 (manufactured by BASF SE) represented by the formula described below was also compounded as the photopolymerization initiator to prepare a film forming material for lithography.

To 5 parts by mass of the film forming material for lithography, 95 parts by mass of PGMEA as a solvent was added, and the resultant mixture was stirred with a stirrer for at least 3 hours or longer at room temperature to prepare a composition for film formation for lithography.

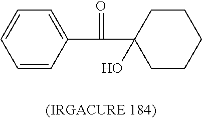 [SMILES: O=C(c1ccccc1)C1(O)CCCCC1]

### Example 21

In addition to 5 parts by mass of the APB-N citramaleimide, 0.1 parts by mass of IRGACURE 184 (manufactured by BASF SE) was also compounded as the photopolymerization initiator, thereby preparing a film forming material for lithography.

The same operations as in the above Example 20 were carried out to prepare a composition for film formation for lithography.

### Example 22

In addition to 5 parts by mass of the HFBAPP citramaleimide, 0.1 parts by mass of IRGACURE 184 (manufactured by BASF SE) was also compounded as the photopolymerization initiator, thereby preparing a film forming material for lithography.

The same operations as in the above Example 20 were carried out to prepare a composition for film formation for lithography.

### Example 23

In addition to 5 parts by mass of the BisAP citramaleimide, 0.1 parts by mass of IRGACURE 184 (manufactured by BASF SE) was also compounded as the photopolymerization initiator, thereby preparing a film forming material for lithography.

The same operations as in the above Example 20 were carried out to prepare a composition for film formation for lithography.

### Example 24

In addition to 5 parts by mass of the BMI citramaleimide resin, 0.1 parts by mass of IRGACURE 184 (manufactured by BASF SE) was also compounded as the photopolymerization initiator, thereby preparing a film forming material for lithography.

The same operations as in the above Example 20 were carried out to prepare a composition for film formation for lithography.

### Example 24A

In addition to 5 parts by mass of the high molecular weight BMI citramaleimide polymer, 0.1 parts by mass of IRGACURE 184 (manufactured by BASF SE) was also compounded as the photopolymerization initiator, thereby preparing a film forming material for lithography.

The same operations as in the above Example 20 were carried out to prepare a composition for film formation for lithography.

### Example 25

In addition to 5 parts by mass of the BAN citramaleimide resin, 0.1 parts by mass of IRGACURE 184 (manufactured by BASF SE) was also compounded as the photopolymerization initiator, thereby preparing a film forming material for lithography.

The same operations as in the above Example 20 were carried out to prepare a composition for film formation for lithography.

### Example 25A

In addition to 5 parts by mass of the high molecular weight BAN citramaleimide polymer, 0.1 parts by mass of IRGACURE 184 (manufactured by BASF SE) was also compounded as the photopolymerization initiator, thereby preparing a film forming material for lithography.

The same operations as in the above Example 20 were carried out to prepare a composition for film formation for lithography.

### Example 26

In addition to 5 parts by mass of the BAPP citramaleimide, 2 parts by mass of BF-BXZ was also used as the crosslinking agent and 0.1 parts by mass of IRGACURE 184 (manufactured by BASF SE) was compounded as the photo-radical polymerization initiator to prepare a film forming material for lithography.

The same operations as in the above Example 20 were carried out to prepare a composition for film formation for lithography.

### Example 27

In addition to 5 parts by mass of the BAPP citramaleimide, 2 parts by mass of NC-3000-L was also used as the crosslinking agent and 0.1 parts by mass of IRGACURE 184 (manufactured by BASF SE) was compounded as the photo-radical polymerization initiator to prepare a film forming material for lithography.

The same operations as in the above Example 20 were carried out to prepare a composition for film formation for lithography.

### Example 28

In addition to 5 parts by mass of the BAPP citramaleimide, 2 parts by mass of DABPA-CN was also used as the crosslinking agent and 0.1 parts by mass of IRGACURE 184 (manufactured by BASF SE) was compounded as the photo-radical polymerization initiator to prepare a material for film formation for lithography.

The same operations as in the above Example 20 were carried out to prepare a composition for film formation for lithography.

### Example 29

In addition to 5 parts by mass of the BAPP citramaleimide, 2 parts by mass of BPA-CA was also used as the crosslinking agent and 0.1 parts by mass of IRGACURE 184 (manufactured by BASF SE) was compounded as the photo-radical polymerization initiator to prepare a film forming material for lithography.

The same operations as in the above Example 20 were carried out to prepare a composition for film formation for lithography.

### Example 30

In addition to 5 parts by mass of the BAPP citramaleimide, 2 parts by mass of APG-1 was also used as the crosslinking agent and 0.1 parts by mass of IRGACURE 184 (manufactured by BASF SE) was compounded as the photo-radical polymerization initiator to prepare a film forming material for lithography.

The same operations as in the above Example 20 were carried out to prepare a composition for film formation for lithography.

### Example 31

In addition to 5 parts by mass of the BAPP citramaleimide, 2 parts by mass of APG-2 was also used as the crosslinking agent and 0.1 parts by mass of IRGACURE 184 (manufactured by BASF SE) was compounded as the photo-radical polymerization initiator to prepare a film forming material for lithography.

The same operations as in the above Example 20 were carried out to prepare a composition for film formation for lithography.

### Example 32

In addition to 5 parts by mass of the BAPP citramaleimide, 2 parts by mass of DDM was also used as the crosslinking agent and 0.1 parts by mass of IRGACURE 184 (manufactured by BASF SE) was compounded as the photo-radical polymerization initiator to prepare a film forming material for lithography.

The same operations as in the above Example 20 were carried out to prepare a composition for film formation for lithography.

### Example 20-2

In addition to 5 parts by mass of the BAPP citramaleimide, 0.1 parts by mass of WPBG-300 (manufactured by FUJIFILM Wako Pure Chemical Corporation) represented by the formula described below was also compounded as the photobase generating agent to prepare a film forming material for lithography.

To 5 parts by mass of the film forming material for lithography, 95 parts by mass of PGMEA as a solvent was added, and the resultant mixture was stirred with a stirrer for at least 3 hours or longer at room temperature, thereby preparing a composition for film formation for lithography.

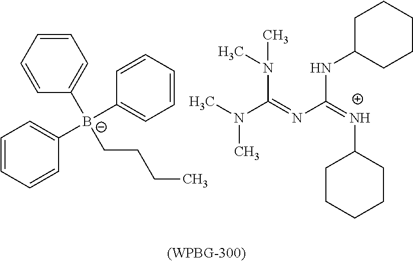 [SMILES: CCCC[B-](c1ccccc1)(c1ccccc1)c1ccccc1 ; CN(C)C(=N/C(NC1CCCCC1)=[NH+]\C1CCCCC1)N(C)C]

### Example 21-2

In addition to 5 parts by mass of the APB-N citramaleimide, 0.1 parts by mass of WPBG-300 (manufactured by FUJIFILM Wako Pure Chemical Corporation) was also compounded as the photobase generating agent to prepare a film forming material for lithography.

The same operations as in the above Example 20 were carried out to prepare a composition for film formation for lithography.

### Example 22-2

In addition to 5 parts by mass of the HFBAPP citramaleimide, 0.1 parts by mass of WPBG-300 (manufactured by FUJIFILM Wako Pure Chemical Corporation) was also compounded as the photobase generating agent to prepare a film forming material for lithography.

The same operations as in the above Example 20 were carried out to prepare a composition for film formation for lithography.

### Example 23-2

In addition to 5 parts by mass of the BisAP citramaleimide, 0.1 parts by mass of WPBG-300 (manufactured by FUJIFILM Wako Pure Chemical Corporation) was also compounded as the photobase generating agent to prepare a film forming material for lithography.

The same operations as in the above Example 20 were carried out to prepare a composition for film formation for lithography.

### Example 24-2

In addition to 5 parts by mass of the BMI citramaleimide resin, 0.1 parts by mass of WPBG-300 (manufactured by FUJIFILM Wako Pure Chemical Corporation) was also compounded as the photobase generating agent to prepare a film forming material for lithography.

The same operations as in the above Example 20 were carried out to prepare a composition for film formation for lithography.

### Example 24A-2

In addition to 5 parts by mass of the high molecular weight BMI citramaleimide polymer, 0.1 parts by mass of WPBG-300 (manufactured by FUJIFILM Wako Pure Chemical Corporation) was also compounded as the photobase generating agent to prepare a film forming material for lithography.

The same operations as in the above Example 20 were carried out to prepare a composition for film formation for lithography.

### Example 25-2

In addition to 5 parts by mass of the BAN citramaleimide resin, 0.1 parts by mass of WPBG-300 (manufactured by FUJIFILM Wako Pure Chemical Corporation) was also compounded as the photobase generating agent to prepare a film forming material for lithography.

The same operations as in the above Example 20 were carried out to prepare a composition for film formation for lithography.

### Example 25A-2

In addition to 5 parts by mass of the high molecular weight BAN citramaleimide polymer, 0.1 parts by mass of WPBG-300 (manufactured by FUJIFILM Wako Pure Chemical Corporation) was also compounded as the photobase generating agent to prepare a film forming material for lithography.

The same operations as in the above Example 20 were carried out to prepare a composition for film formation for lithography.

### Example 26-2

In addition to 5 parts by mass of the BAPP citramaleimide, 2 parts by mass of BF-BXZ was also used as the crosslinking agent and 0.1 parts by mass of WPBG-300 (manufactured by FUJIFILM Wako Pure Chemical Corporation) was compounded as the photobase generating agent to prepare a film forming material for lithography.

The same operations as in the above Example 20 were carried out to prepare a composition for film formation for lithography.

### Example 27-2

In addition to 5 parts by mass of the BAPP citramaleimide, 2 parts by mass of NC-3000-L was also used as the crosslinking agent and 0.1 parts by mass of WPBG-300 (manufactured by FUJIFILM Wako Pure Chemical Corporation) was compounded as the photobase generating agent to prepare a film forming material for lithography.

The same operations as in the above Example 20 were carried out to prepare a composition for film formation for lithography.

### Example 28-2

In addition to 5 parts by mass of the BAPP citramaleimide, 2 parts by mass of DABPA-CN was also used as the crosslinking agent and 0.1 parts by mass of WPBG-300 (manufactured by FUJIFILM Wako Pure Chemical Corporation) was compounded as the photobase generating agent to prepare a material for film formation for lithography.

The same operations as in the above Example 20 were carried out to prepare a composition for film formation for lithography.

### Example 29-2

In addition to 5 parts by mass of the BAPP citramaleimide, 2 parts by mass of BPA-CA was also used as the crosslinking agent and 0.1 parts by mass of IRGACURE 184 (manufactured by BASF SE) was compounded as the photo-radical polymerization initiator, thereby preparing a film forming material for lithography.

The same operations as in the above Example 20 were carried out to prepare a composition for film formation for lithography.

### Example 30-2

In addition to 5 parts by mass of the BAPP citramaleimide, 2 parts by mass of APG-1 was also used as the crosslinking agent and 0.1 parts by mass of WPBG-300 (manufactured by FUJIFILM Wako Pure Chemical Corporation) was compounded as the photobase generating agent to prepare a film forming material for lithography.

The same operations as in the above Example 20 were carried out to prepare a composition for film formation for lithography.

### Example 31-2

In addition to 5 parts by mass of the BAPP citramaleimide, 2 parts by mass of APG-2 was also used as the crosslinking agent and 0.1 parts by mass of WPBG-300 (manufactured by FUJIFILM Wako Pure Chemical Corporation) was compounded as the photobase generating agent to prepare a film forming material for lithography.

The same operations as in the above Example 20 were carried out to prepare a composition for film formation for lithography.

### Example 32-2

In addition to 5 parts by mass of the BAPP citramaleimide, 2 parts by mass of DDM was also used as the crosslinking agent and 0.1 parts by mass of WPBG-300 (manufactured by FUJIFILM Wako Pure Chemical Corporation) was compounded as the photobase generating agent to prepare a film forming material for lithography.

The same operations as in the above Example 20 were carried out to prepare a composition for film formation for lithography.

<Preparation of underlayer film from compositions for film formation for lithography of Examples 1 to 19 and Comparative Examples 1 to 10>

A silicon substrate was spin coated with each of the compositions for film formation for lithography of Examples 1 to 19 and Comparative Examples 1 to 10, which have the compositions shown in Table 1, and then baked at 240° C. for 60 seconds. Then, the film thickness of the resultant coated film was measured. Thereafter, the silicon substrate was immersed in a mixed solvent of 70% PGMEA/30% PGME for 60 seconds, the adhered solvent was removed with an Aero Duster, and then the substrate was subjected to solvent drying at 110° C. From the difference in film thickness before and after the immersion, the decreasing rate of film thickness (%) was calculated, and the curability of each underlayer film was evaluated under the evaluation criteria shown below.

The underlayer films after the curing baking at 240° C. were further baked at 400° C. for 120 seconds, and from the difference in film thickness before and after the baking, the decreasing rate of film thickness (%) was calculated and the film heat resistance of each underlayer film was evaluated under the evaluation criteria shown below. Then, under the conditions shown below, the etching resistance was evaluated.

In addition, the embedding properties to a substrate having difference in level and the flatness were evaluated under the conditions shown below. <Preparation of underlayer film from compositions for film formation for lithography of Examples 20 to 32 and Examples 20-2 to 32-2>

A silicon substrate was spin coated with each of the compositions for film formation for lithography of Examples 20 to 32, and 20-2 to 32-2, which have the compositions shown in Table 2, and then baked at 150° C. for 60 seconds to remove the solvent in the coated film. Subsequently, the film was cured using a high pressure mercury lamp with an accumulated light exposure of 1500 mJ/cm² and an irradiation time of 60 seconds, and then the film thickness of the coated film was measured. Thereafter, the silicon substrate was immersed in a mixed solvent of 70% PGMEA/30% PGME for 60 seconds, the adhered solvent was removed with an Aero Duster, and then the substrate was subjected to solvent drying at 110° C. From the difference in film thickness before and after the immersion, the decreasing rate of film thickness (%) was calculated and the curability of each underlayer film was evaluated under the evaluation criteria shown below.

The underlayer films were further baked at 400° C. for 120 seconds, and from the difference in film thickness before and after the baking, the decreasing rate of film thickness (%) was calculated and the film heat resistance of each underlayer film was evaluated under the evaluation criteria shown below. Then, under the conditions shown below, the etching resistance was evaluated.

In addition, the embedding properties to a substrate having difference in level and the flatness were evaluated under the conditions shown below.

### [Evaluation of Curability]

### <Evaluation Criteria>

S: Decreasing rate of film thickness before and after solvent immersion ≤1%

A: 1%<Decreasing rate of film thickness before and after solvent immersion ≤5%

B: Decreasing rate of film thickness before and after solvent immersion>5%

### [Evaluation of Film Heat Resistance]

### <Evaluation Criteria>

S: Decreasing rate of film thickness before and after baking at 400° C. ≤10%

A: 10%<Decreasing rate of film thickness before and after baking at 400° C. ≤15%

B: 15%<Decreasing rate of film thickness before and after baking at 400° C. ≤20%

C: Decreasing rate of film thickness before and after baking at 400° C.>20%

### [Etching Test]

Etching apparatus: RIE-10NR manufactured by Samco International, Inc.

Output: 50 W

Pressure: 4 Pa

Time: 2 min

Etching gas

CF₄ gas flow rate:O₂ gas flow rate=5:15 (sccm)

### [Evaluation of Etching Resistance]

The evaluation of etching resistance was carried out by the following procedures.

First, an underlayer film of novolac was prepared under the same conditions as in Example 1 except that novolac (PSM 4357 manufactured by Gun Ei Chemical Industry Co., Ltd.) was used instead of the film forming material for lithography in Example 1 and the drying temperature was 110° C. Then, this underlayer film of novolac was subjected to the etching test mentioned above, and the etching rate was measured.

Next, the underlayer films of Examples 1 to 19 and Comparative Examples 1 to 10 were subjected to the etching test described above in the same way as above, and the etching rate was measured.

Then, the etching resistance was evaluated according to the following evaluation criteria on the basis of the etching rate of the underlayer film of novolac. From a practical viewpoint, evaluation S described below is particularly preferable, and evaluation A and evaluation B are preferable.

### <Evaluation Criteria>

S: The etching rate was less than −30% as compared with the underlayer film of novolac.
A: The etching rate was −30% or more to less than −20% as compared with the underlayer film of novolac.
B: The etching rate was −20% or more to less than −10% as compared with the underlayer film of novolac.
C: The etching rate was −10% or more and 0% or less as compared with the underlayer film of novolac.

### [Evaluation of Embedding Properties to Substrate Having Difference in Level]

The embedding properties to a substrate having difference in level were evaluated by the following procedures.

A SiO₂ substrate having a film thickness of 80 nm and a line and space pattern of 60 nm was coated with a composition for underlayer film formation for lithography, and baked at 240° C. for 60 seconds to form a 90 nm underlayer film. The cross section of the obtained film was cut out and observed under an electron microscope to evaluate the embedding properties to a substrate having difference in level.

### <Evaluation Criteria>

A: The underlayer film was embedded without defects in the asperities of the SiO₂ substrate having a line and space pattern of 60 nm.

C: The asperities of the SiO2 substrate having a line and space pattern of 60 nm had defects which hindered the embedding of the underlayer film.

### [Evaluation of Flatness]

Onto a SiO₂ substrate having difference in level on which trenches with a width of 100 nm, a pitch of 150 nm, and a depth of 150 nm (aspect ratio: 1.5) and trenches with a width of 5 μm and a depth of 180 nm (open space) were mixedly present, each of the obtained compositions for film formation was coated. Subsequently, it was calcined at 240° C. for 120 seconds under the air atmosphere to form a resist underlayer film having a film thickness of 200 nm. The shape of this resist underlayer film was observed with a scanning electron microscope (“S-4800” from Hitachi High-Technologies Corporation), and the difference between the maximum value and the minimum value of the film thickness of the resist underlayer film on the trench or space (ΔFT) was measured.

### <Evaluation Criteria>

S: ΔFT<10 nm (best flatness)

A: 10 nm≤ΔFT<20 nm (good flatness)

B: 20 nm≤ΔFT<40 nm (partially good flatness)

C: 40 nm≤ΔFT (poor flatness)

TABLE 1

| | | | Crosslinking | | | | | | |
|---|---|---|---|---|---|---|---|---|---|
| | Maleimide | Crosslinking | promoting | | | Film heat | Etching | Embedding | |
| | compound | agent | agent | Solvent | Curability | resistance | resistance | properties | Flatness |
| Example 1 | BAPP | — | — | PGMEA(95) | S | A | S | A | S |
| | citramaleimide | | | | | | | | |
| | (5) | | | | | | | | |
| Example 2 | APB-N | — | — | PGMEA(95) | S | A | S | A | S |
| | citramaleimide | | | | | | | | |
| | (5) | | | | | | | | |
| Example 3 | HFBAPP | — | — | PGMEA(95) | S | A | S | A | S |
| | citramaleimide | | | | | | | | |
| | (5) | | | | | | | | |
| Example 4 | BisAP | — | — | PGMEA(95) | S | A | S | A | S |
| | citramaleimide | | | | | | | | |
| | (5) | | | | | | | | |
| Example 5 | BMI | — | — | PGMEA(95) | A | A | S | A | S |
| | citramaleimide | | | | | | | | |
| | resin (5) | | | | | | | | |
| Example 5A | High | — | — | PGMEA(95) | A | S | S | A | S |
| | molecular | | | | | | | | |
| | weight BMI | | | | | | | | |
| | citramaleimide | | | | | | | | |
| | polymer (5) | | | | | | | | |
| Example 6 | BAN | — | — | PGMEA(95) | A | A | A | A | S |
| | citramaleimide | | | | | | | | |
| | resin (5) | | | | | | | | |
| Example 6A | High | — | — | PGMEA(95) | A | S | A | A | S |
| | molecular | | | | | | | | |
| | weight BAN | | | | | | | | |
| | citramaleimide | | | | | | | | |
| | polymer (5) | | | | | | | | |
| Example 7 | BAPP | — | TPIZ | PGMEA(95) | S | S | S | A | S |
| | citramaleimide | | (0.1) | | | | | | |
| | (5) | | | | | | | | |
| Example 8 | APB-N | — | TPIZ | PGMEA(95) | S | S | S | A | S |
| | citramaleimide | | (0.1) | | | | | | |
| | (5) | | | | | | | | |
| Example 9 | HFBAPP | — | TPIZ | PGMEA(95) | S | S | S | A | S |
| | citramaleimide | | (0.1) | | | | | | |
| | (5) | | | | | | | | |
| Example 10 | BisAP | — | TPIZ | PGMEA(95) | S | S | S | A | S |
| | citramaleimide | | (0.1) | | | | | | |
| | (5) | | | | | | | | |
| Example 11 | BMI | — | TPIZ | PGMEA(95) | S | A | S | A | S |
| | citramaleimide | | (0.1) | | | | | | |
| | resin (5) | | | | | | | | |
| Example 11A | High | — | TPIZ | PGMEA(95) | S | S | S | A | S |
| | molecular | | (0.1) | | | | | | |
| | weight BMI | | | | | | | | |
| | citramaleimide | | | | | | | | |
| | polymer (5) | | | | | | | | |
| Example 12 | BAN | — | TPIZ | PGMEA(95) | S | A | S | A | S |
| | citramaleimide | | (0.1) | | | | | | |
| | resin (5) | | | | | | | | |
| Example 12A | High | — | TPIZ | PGMEA(95) | S | S | S | A | S |
| | molecular | | (0.1) | | | | | | |
| | weight BAN | | | | | | | | |
| | citramaleimide | | | | | | | | |
| | polymer (5) | | | | | | | | |
| Example 13 | BAPP | BF-BXZ(2) | TPIZ | PGMEA(95) | S | S | S | A | S |
| | citramaleimide | | (0.1) | | | | | | |
| | (5) | | | | | | | | |
| Example 14 | BAPP | NC-3000- | TPIZ | PGMEA(95) | S | S | S | A | S |
| | citramaleimide | L(2) | (0.1) | | | | | | |
| | (5) | | | | | | | | |
| Example 15 | BAPP | DABPA- | TPIZ | PGMEA(95) | S | S | S | A | S |
| | citramaleimide | CN | (0.1) | | | | | | |
| | (5) | (2) | | | | | | | |
| Example 16 | BAPP | BPA-CN | TPIZ | PGMEA(95) | S | S | S | A | S |
| | citramaleimide | (2) | (0.1) | | | | | | |
| | (5) | | | | | | | | |
| Example 17 | BAPP | APG-1 | — | PGMEA(95) | S | S | S | A | S |
| | citramaleimide | (2) | | | | | | | |
| | (5) | | | | | | | | |
| Example 18 | BAPP | APG-2 | — | PGMEA(95) | S | S | S | A | S |
| | citramaleimide | (2) | | | | | | | |
| | (5) | | | | | | | | |
| Example 19 | BAPP | DDM | — | PGMEA(95) | S | S | S | A | S |
| | citramaleimide | (2) | | | | | | | |
| | (5) | | | | | | | | |
| Comparative | CR-1 | NC-3000- | TPIZ | PGMEA(95) | A | C | C | C | C |
| Example 1 | (5) | L(2) | (0.1) | | | | | | |
| Comparative | CR-1 | — | — | PGMEA(95) | A | C | C | C | C |
| Example 2 | (5) | | | | | | | | |
| Comparative | BAPP | — | — | PGMEA(95) | B | B | A | A | S |
| Example 3 | citraconimide | | | | | | | | |
| | (5) | | | | | | | | |
| Comparative | APB-N | — | — | PGMEA(95) | B | B | S | A | S |
| Example 4 | citraconimide | | | | | | | | |
| | (5) | | | | | | | | |
| Comparative | HFBAPP | — | — | PGMEA(95) | B | B | S | A | S |
| Example 5 | citraconimide | | | | | | | | |
| | (5) | | | | | | | | |
| Comparative | BisAP | — | — | PGMEA(95) | B | B | A | A | S |
| Example 6 | citraconimide | | | | | | | | |
| | (5) | | | | | | | | |
| Comparative | BMI | — | — | PGMEA(95) | B | B | A | A | S |
| Example 7 | citraconimide | | | | | | | | |
| | resin (5) | | | | | | | | |
| Comparative | BAN | — | — | PGMEA(95) | B | B | B | A | S |
| Example 8 | citraconimide | | | | | | | | |
| | resin (5) | | | | | | | | |
| Comparative | BMI-2300(5) | — | — | PGMEA(95) | S | A | A | A | B |
| Example 9 | | | | | | | | | |
| Comparative | BMI-80(5) | — | — | PGMEA(95) | S | A | A | A | A |
| Example 10 | | | | | | | | | |

As is evident from Table 1, it was confirmed that Examples 1 to 19, in which the compositions for film formation for lithography of the present embodiment comprising citramaleimides and citramaleimide resins were used, have superior curability, film heat resistance, and etching resistance compared to the citraconimides of Comparative Examples 3 to 8, and superior flatness compared to the maleimides of Comparative Examples 9 to 10. In particular, it was confirmed that the use of high molecular weight BMI citramaleimide polymer or high molecular weight BAN citramaleimide polymer achieves both high film heat resistance and excellent flatness.

In addition, the compositions of Examples 1 to 6 and Comparative Examples 3 to 10 were subjected to a storage stability test at room temperature of 25° C. for one month, and the presence or absence of precipitates was visually confirmed. As a result, it was confirmed that there is no precipitation in the compositions of Examples 1 to 6, but precipitates were visually confirmed in the compositions of Comparative Examples 3 to 10.

Accordingly, it was confirmed that the compositions for film formation for lithography of the present embodiment comprising citramaleimides and citramaleimide resins have superior solvent solubility and storage stability compared to the citraconimides of Comparative Examples 3 to 8 and the maleimides of Comparative Examples 9 to 10.

TABLE 2

| | | | Radical | | | | | | |
|---|---|---|---|---|---|---|---|---|---|
| | Maleimide | Crosslinking | polymerization | | | Film heat | Etching | Embedding | |
| | compound | agent | initiator | Solvent | Curability | resistance | resistance | properties | Flatness |
| Example 20 | BAPP | — | IRGACURE184 | PGMEA(95) | S | A | B | A | S |
| | citramaleimide | | (0.1) | | | | | | |
| | (5) | | | | | | | | |
| Example 21 | APB-N | — | IRGACURE184 | PGMEA(95) | S | A | B | A | S |
| | citramaleimide | | (0.1) | | | | | | |
| | (5) | | | | | | | | |
| Example 22 | HFBAPP | — | IRGACURE184 | PGMEA(95) | S | A | B | A | S |
| | citramaleimide | | (0.1) | | | | | | |
| | (5) | | | | | | | | |
| Example 23 | BisAP | — | IRGACURE184 | PGMEA(95) | S | A | B | A | S |
| | citramaleimide | | (0.1) | | | | | | |
| | (5) | | | | | | | | |
| Example 24 | BMI | — | IRGACURE184 | PGMEA(95) | A | A | B | A | S |
| | citramaleimide | | (0.1) | | | | | | |
| | resin (5) | | | | | | | | |
| Example 24A | High | — | IRGACURE184 | PGMEA(95) | A | S | B | A | S |
| | molecular | | (0.1) | | | | | | |
| | weight BMI | | | | | | | | |
| | citramaleimide | | | | | | | | |
| | polymer (5) | | | | | | | | |
| Example 25 | BAN | — | IRGACURE184 | PGMEA(95) | A | A | B | A | S |
| | citramaleimide | | (0.1) | | | | | | |
| | resin (5) | | | | | | | | |
| Example 25A | High | — | IRGACURE184 | PGMEA(95) | A | S | B | A | S |
| | molecular | | (0.1) | | | | | | |
| | weight BAN | | | | | | | | |
| | citramaleimide | | | | | | | | |
| | polymer (5) | | | | | | | | |
| Example 26 | BAPP | BF-BXZ(2) | IRGACURE184 | PGMEA(95) | S | S | S | A | S |
| | citramaleimide | | (0.1) | | | | | | |
| | (5) | | | | | | | | |
| Example 27 | BAPP | NC-3000- | IRGACURE184 | PGMEA(95) | S | S | S | A | S |
| | citramaleimide | L(2) | (0.1) | | | | | | |
| | (5) | | | | | | | | |
| Example 28 | BAPP | DABPA- | IRGACURE184 | PGMEA(95) | S | S | S | A | S |
| | citramaleimide | CN(2) | (0.1) | | | | | | |
| | (5) | | | | | | | | |
| Example 29 | BAPP | BPA-CN(2) | IRGACURE184 | PGMEA(95) | S | S | S | A | S |
| | citramaleimide | | (0.1) | | | | | | |
| | (5) | | | | | | | | |
| Example 30 | BAPP | APG-1(2) | IRGACURE184 | PGMEA(95) | S | S | S | A | S |
| | citramaleimide | | (0.1) | | | | | | |
| | (5) | | | | | | | | |
| Example 31 | BAPP | APG-2(2) | IRGACURE184 | PGMEA(95) | S | S | S | A | S |
| | citramaleimide | | (0.1) | | | | | | |
| | (5) | | | | | | | | |
| Example 32 | BAPP | DDM(2) | IRGACURE184 | PGMEA(95) | S | S | S | A | S |
| | citramaleimide | | (0.1) | | | | | | |
| | (5) | | | | | | | | |
| Example 20-2 | BAPP | — | WPBG-300 | PGMEA(95) | S | A | B | A | S |
| | citramaleimide | | (0.1) | | | | | | |
| | (5) | | | | | | | | |
| Example 21-2 | APB-N | — | WPBG-300 | PGMEA(95) | S | A | B | A | S |
| | citramaleimide | | (0.1) | | | | | | |
| | (5) | | | | | | | | |
| Example 22-2 | HFBAPP | — | WPBG-300 | PGMEA(95) | S | A | B | A | S |
| | citramaleimide | | (0.1) | | | | | | |
| | (5) | | | | | | | | |
| Example 23-2 | BisAP | — | WPBG-300 | PGMEA(95) | S | A | B | A | S |
| | citramaleimide | | (0.1) | | | | | | |
| | (5) | | | | | | | | |
| Example 24-2 | BMI | — | WPBG-300 | PGMEA(95) | S | S | B | A | S |
| | citramaleimide | | (0.1) | | | | | | |
| | resin (5) | | | | | | | | |
| Example 24A-2 | High | — | WPBG-300 | PGMEA(95) | S | S | B | A | S |
| | molecular | | (0.1) | | | | | | |
| | weight BMI | | | | | | | | |
| | citramaleimide | | | | | | | | |
| | polymer (5) | | | | | | | | |
| Example 25-2 | BAN | — | WPBG-300 | PGMEA(95) | S | S | B | A | S |
| | citramaleimide | | (0.1) | | | | | | |
| | resin (5) | | | | | | | | |
| Example 25A-2 | High | — | WPBG-300 | PGMEA(95) | S | S | B | A | S |
| | molecular | | (0.1) | | | | | | |
| | weight BAN | | | | | | | | |
| | citramaleimide | | | | | | | | |
| | polymer (5) | | | | | | | | |
| Example 26-2 | BAPP | BF-BXZ(2) | WPBG-300 | PGMEA(95) | S | S | S | A | S |
| | citramaleimide | | (0.1) | | | | | | |
| | (5) | | | | | | | | |
| Example 27-2 | BAPP | NC-3000- | WPBG-300 | PGMEA(95) | S | S | S | A | S |
| | citramaleimide | L(2) | (0.1) | | | | | | |
| | (5) | | | | | | | | |
| Example 28-2 | BAPP | DABPA- | WPBG-300 | PGMEA(95) | S | S | S | A | S |
| | citramaleimide | CN(2) | (0.1) | | | | | | |
| | (5) | | | | | | | | |
| Example 29-2 | BAPP | BPA-CN(2) | WPBG-300 | PGMEA(95) | S | S | S | A | S |
| | citramaleimide | | (0.1) | | | | | | |
| | (5) | | | | | | | | |
| Example 30-2 | BAPP | APG-1(2) | WPBG-300 | PGMEA(95) | S | S | S | A | S |
| | citramaleimide | | (0.1) | | | | | | |
| | (5) | | | | | | | | |
| Example 31-2 | BAPP | APG-2(2) | WPBG-300 | PGMEA(95) | S | S | S | A | S |
| | citramaleimide | | (0.1) | | | | | | |
| | (5) | | | | | | | | |
| Example 32-2 | BAPP | DDM(2) | WPBG-300 | PGMEA(95) | S | S | S | A | S |
| | citramaleimide | | (0.1) | | | | | | |
| | (5) | | | | | | | | |

### Example 33

A SiO₂ substrate with a film thickness of 300 nm was coated with the composition for film formation for lithography in Example 1, and baked at 240° C. for 60 seconds and further at 400° C. for 120 seconds to form an underlayer film with a film thickness of 70 nm. This underlayer film was coated with a resist solution for ArF and baked at 130° C. for 60 seconds to form a photoresist layer with a film thickness of 140 nm. The resist solution for ArF used was prepared by compounding 5 parts by mass of a compound of the following formula (22), 1 part by mass of triphenylsulfonium nonafluoromethanesulfonate, 2 parts by mass of tributylamine, and 92 parts by mass of PGMEA.

Note that the compound of the following formula (22) was prepared as follows. That is, 4.15 g of 2-methyl-2-methacryloyloxyadamantane, 3.00 g of methacryloyloxy-γ-butyrolactone, 2.08 g of 3-hydroxy-1-adamantyl methacrylate, and 0.38 g of azobisisobutyronitrile were dissolved in 80 mL of tetrahydrofuran to prepare a reaction solution. This reaction solution was polymerized for 22 hours with the reaction temperature kept at 63° C. in a nitrogen atmosphere. Then, the reaction solution was added dropwise into 400 mL of n-hexane. The product resin thus obtained was solidified and purified, and the resulting white powder was filtered and dried overnight at 40° C. under reduced pressure to obtain a compound represented by the following formula.

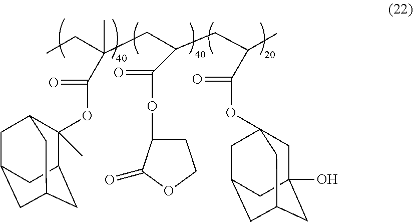

In the above formula (22), 40, 40, and 20 represent the ratio of each constituent unit and do not represent a block copolymer.

Subsequently, the photoresist layer was exposed using an electron beam lithography system (manufactured by ELIONIX INC.; ELS-7500, 50 keV), baked (PEB) at 115° C. for 90 seconds, and developed for 60 seconds in a 2.38 mass % tetramethylammonium hydroxide (TMAH) aqueous solution to obtain a positive type resist pattern. The evaluation results are shown in Table 3.

### Example 34

A positive type resist pattern was obtained in the same way as Example 33 except that the composition for underlayer film formation for lithography in Example 2 was used instead of the composition for underlayer film formation for lithography in the above Example 1. The evaluation results are shown in Table 3.

### Example 35

A positive type resist pattern was obtained in the same way as Example 33 except that the composition for underlayer film formation for lithography in Example 3 was used instead of the composition for underlayer film formation for lithography in the above Example 1. The evaluation results are shown in Table 3.

### Example 36

A positive type resist pattern was obtained in the same way as Example 33 except that the composition for underlayer film formation for lithography in Example 4 was used instead of the composition for underlayer film formation for lithography in the above Example 1. The evaluation results are shown in Table 3.

### Comparative Example 11

The same operations as in Example 33 were performed except that no underlayer film was formed so that a photoresist layer was formed directly on a SiO₂ substrate to obtain a positive type resist pattern. The evaluation results are shown in Table 3.

### [Evaluation]

Concerning each of Examples 33 to 36 and Comparative Example 11, the shapes of the obtained 55 nm L/S (1:1) and 80 nm L/S (1:1) resist patterns were observed under an electron microscope (S-4800) manufactured by Hitachi, Ltd. The shapes of the resist patterns after development were evaluated as goodness when having good rectangularity without pattern collapse, and as poorness if this was not the case. The smallest line width having good rectangularity without pattern collapse as a result of this observation was used as an index for resolution evaluation. The smallest electron beam energy quantity capable of lithographing good pattern shapes was used as an index for sensitivity evaluation.

TABLE 3

| | Composition | | | |
|---|---|---|---|---|
| | for film | | | Resist pattern |
| | formation for | Resolution | Sensitivity | shape after |
| | lithography | (nm L/S) | (μC/cm²) | development |
| Example 33 | Composition | 53 | 17 | Goodness |
| | described in | | | |
| | Example 1 | | | |
| Example 34 | Composition | 61 | 17 | Goodness |
| | described in | | | |
| | Example 2 | | | |
| Example 35 | Composition | 54 | 16 | Goodness |
| | described in | | | |
| | Example 3 | | | |
| Example 36 | Composition | 48 | 16 | Goodness |
| | described in | | | |
| | Example 4 | | | |
| Comparative | — | 90 | 41 | Poorness |
| Example 11 | | | | |

As is evident from Table 3, it was confirmed that Examples 33 to 36, in which the compositions for film formation for lithography of the present embodiment comprising citramaleimides and citramaleimide resins were used, are significantly superior in both resolution and sensitivity to Comparative Example 11. Also, the resist pattern shapes after development were confirmed to have good rectangularity without pattern collapse. Furthermore, the difference in the resist pattern shapes after development indicated that the underlayer films of Examples 33 to 36 obtained from the compositions for film formation for lithography of Examples 1, 2, 3, and 4 have good adhesiveness to a resist material.

The present application is based on Japanese Patent Application No. 2018-218042 filed on November 21, 2018, the contents of which are incorporated herein by reference.

### INDUSTRIAL APPLICABILITY

The film forming material for lithography of the present embodiment has relatively high heat resistance, relatively high solvent solubility, and excellent embedding properties to a substrate having difference in level and film flatness, and is applicable to a wet process. Therefore, the composition for film formation for lithography comprising the film forming material for lithography can be utilized widely and effectively in various applications that require such performances. In particular, the present invention can be utilized particularly effectively in the field of underlayer films for lithography and underlayer films for multilayer resist.

1. A film forming material for lithography comprising a compound having:
a group of formula (0A): 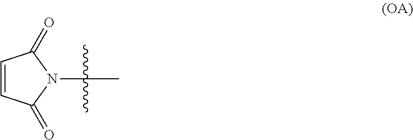 [SMILES: *N1C(=O)C=CC1=O]
and
a group of formula (0B): 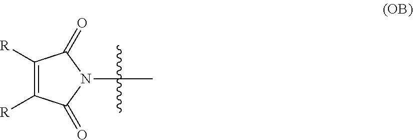
wherein
each R is independently selected from the group consisting of a hydrogen atom and an alkyl group having 1 to 4 carbon atoms, provided that at least one R is an alkyl group having 1 to 4 carbon atoms.

2. The film forming material for lithography according to claim 1, wherein the compound is represented by formula (1A₀): 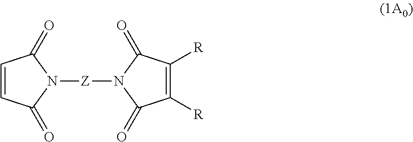
wherein
each R is independently selected from the group consisting of a hydrogen atom and an alkyl group having 1 to 4 carbon atoms, provided that at least one R is an alkyl group having 1 to 4 carbon atoms; and
Z is a divalent group having 1 to 100 carbon atoms and optionally containing a heteroatom.

3. The film forming material for lithography according to claim 1, wherein the compound is represented by formula (1A): 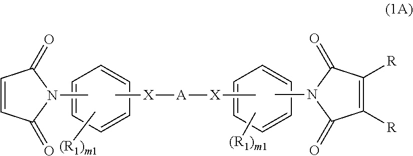
wherein
each R is independently selected from the group consisting of a hydrogen atom and an alkyl group having 1 to 4 carbon atoms, provided that at least one R is an alkyl group having 1 to 4 carbon atoms;
each X is independently selected from the group consisting of a single bond, —O—, —CH₂—, —C(CH₃)₂—, —CO—, —C(CF₃)₂—, —CONH—, and —COO—;
A is selected from the group consisting of a single bond, an oxygen atom, and a divalent group having 1 to 80 carbon atoms and optionally containing a heteroatom;
each R1 is independently a group having 0 to 30 carbon atoms and optionally containing a heteroatom; and
each m1 is independently an integer of 0 to 4.

4. The film forming material for lithography according to claim 3, wherein:
A is a single bond, an oxygen atom, —(CH2)ₚ—, —CH₂C(CH₃)₂CH₂—, —(C(CH₃)₂)ₚ—, —(O(CH₂)_{q})ₚ—, —(O(C₆H4))ₚ—, or any of the following structures: 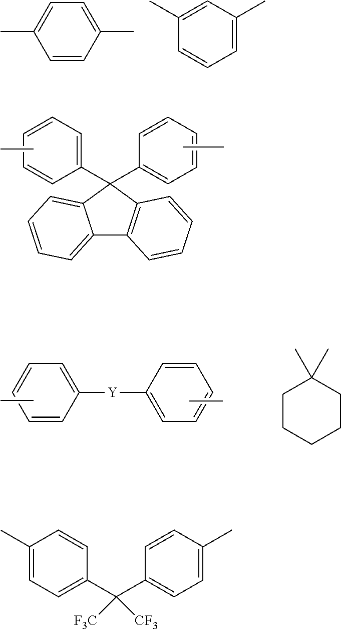
Y is a single bond, —O—, —CH₂—, —C(CH₃)₂—, —C(CF₃)₂—, 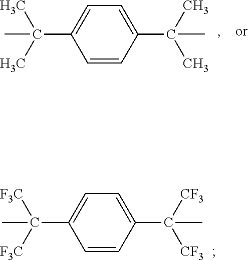
p is an integer of 0 to 20; and
q is an integer of 0 to 4.

5. The film forming material for lithography according to claim 3, wherein:
each X is independently a single bond, —O—, —C(CH₃)₂—, —CO—, or —COO—;
A is a single bond, an oxygen atom, or the following structures: 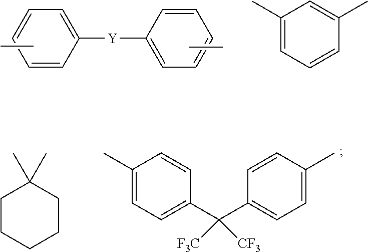
and
Y is —C(CH₃)₂— or —C(CF₃)₂—.

6. The film forming material for lithography according to claim 1, wherein the compound is represented by formula (2A): 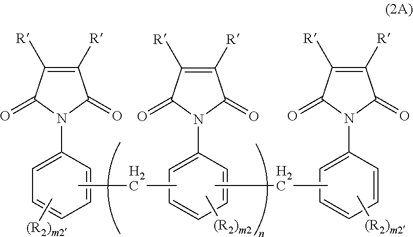
wherein
each R′ is independently selected from the group consisting of a hydrogen atom and an alkyl group having 1 to 4 carbon atoms;
each R₂ is independently a group haying 0 to 10 carbon atoms and optionally containing a heteroatom;
each m2 is independently an integer of 0 to 3;
each m2′ is independently an integer of 0 to 4;
n is an integer of 0 to 4; and
a plurality of groups represented by: 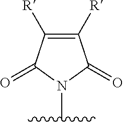
comprise at least a group of formula (0A) and a group of formula (0B).

7. The film forming material for lithography according to claim 1, wherein the compound is represented by formula (3A): 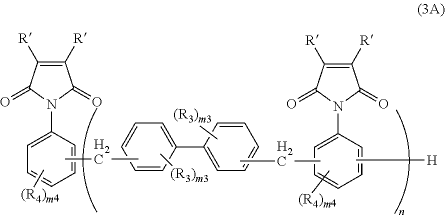
wherein
each R′ is independently selected from the group consisting of a hydrogen atom and an alkyl group having 1 to 4 carbon atoms;
R₃ and R₄ are each independently a group having 0 to 10 carbon atoms and optionally containing a heteroatom;
each m3 is independently an integer of 0 to 4;
each m4 is independently an integer of 0 to 4;
n is an integer of 1 to 4; and
a plurality of groups represented by: 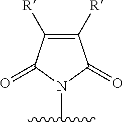
comprise at least a group of formula (0A) and a group of formula (0B).

8. The film forming material for lithography according to claim 2, wherein the heteroatom is selected from the group consisting of oxygen, fluorine, and silicon.

9. The film forming material for lithography according to claim 1, further comprising a crosslinking agent.

10. (canceled)

11. The film forming material for lithography according to claim 9, wherein the crosslinking agent has at least one allyl group.

12. (canceled)

13. The film forming material for lithography according to claim 1, further comprising a crosslinking promoting agent.

14. (canceled)

15. (canceled)

16. The film forming material for lithography according to claim 1, further comprising a radical polymerization initiator.

17. (canceled)

18. (canceled)

19. A composition for film formation for lithography comprising the film forming material for lithography according to claim 1 and a solvent.

20. The composition for film formation for lithography according to claim 19, further comprising an acid generating agent.

21. The composition for film formation for lithography according to claim 19, further comprising a basic compound.

22. The composition for film formation for lithography according to claim 19, wherein the film for lithography is an underlayer film for lithography.

23. An underlayer film for lithography formed by using the composition for film formation for lithography according to claim 22.

24. A method for forming a resist pattern, comprising the steps of:
forming an underlayer film on a substrate by using the composition for film formation for lithography according to claim 22;
forming at least one photoresist layer on the underlayer film; and
irradiating a predetermined region of the photoresist layer with radiation for development.

25. A method for forming a pattern, comprising the steps of:
forming an underlayer film on a substrate by using the composition for film formation for lithography according to claim 22;
forming an intermediate layer film on the underlayer film by using a resist intermediate layer film material containing a silicon atom;
forming at least one photoresist layer on the intermediate layer film;
irradiating a predetermined region of the photoresist layer with radiation for development, thereby forming a resist pattern;
etching the intermediate layer film with the resist pattern as a mask;
etching the underlayer film with the obtained intermediate layer film pattern as an etching mask; and
etching the substrate with the obtained underlayer film pattern as an etching mask, thereby forming a pattern on the substrate.

26. A purification method comprising the steps of:
obtaining an organic phase by dissolving the film forming material for lithography according to claim 1 in a solvent; and
extracting impurities in the film forming material for lithography by bringing the organic phase into contact with an acidic aqueous solution (a first extraction step), wherein
the solvent used in the step of obtaining the organic phase contains a solvent that does not inadvertently mix with water.

27. (canceled)

28. (canceled)

29. (canceled)
